(19) United States
(12) Patent Application Publication
(10) Pub. No.: US 2026/0229352 A1
(43) Pub. Date: Aug. 6, 2026

(54) A NOVEL SYSTEM AND METHOD FOR EARLY-STAGE DETECTION OF MULTIPLE CANCERS

(71) Applicant: PREDOMIX HEALTH SCIENCES PRIVATE LIMITED, Singapore (SG)

(72) Inventors: Kanury RAO VENKATA SUBBA, Haryana Gurgaon (IN); Najmuddin MOHD SAQUIB, Bihar (IN); Zaved SIDDIUI, New Delhi (IN); Ankur GUPTA, Haryana Faridabad (IN)

(73) Assignee: PREDOMIX HEALTH SCIENCES PRIVATE LIMITED, Singapore (SG)

(21) Appl. No.: 19/147,304

(22) PCT Filed: Jan. 11, 2024
(86) PCT No.: PCT/SG2024/050022

§ 371 Date: Jul. 10, 2025

(30) Foreign Application Priority Data
Jan. 11, 2023 (IN) 202311002270

(51) Int. Cl. G16H 50/20; G01N 30/02; G01N 30/88; G01N 33/50; G16B 40/30; G16H 10/40; G16H 10/60

(52) U.S. Cl. CPC G16H 50/20; G01N 30/88; G01N 33/50; G16B 40/30; G16H 10/40; G16H 10/60; G01N 2030/027; G01N 2030/8804; G01N 2560/00; G01N 2800/7028

(57) ABSTRACT

The present invention describes a comprehensive system and method for the simultaneous early detection of multiple cancers in a single analysis. The system involves a Liquid Chromatography-Mass Spectrometry (LC-MS) device coupled with processors and AI/ML algorithms. The LC-MS device analyses metabolite ions from dried extracts of biological fluid samples, aligning and normalizing the data while minimizing errors. Quality control processes, including a neural network model and critical ion monitoring, ensure accurate detection. The system employs AI/ML processes to create two models: the Cancer Detection AI (CDAI) Model for identifying cancerous samples, and the Tissue of Origin Identification (TOOAI) Model for distinguishing specific cancer types. The models are applied to test samples, providing scores based on tissue of origin probabilities. The invention aims to revolutionize early cancer detection through advanced analytical and machine learning techniques.

### TECHNICAL FIELD OF THE INVENTION

The present invention relates to the field of clinical metabolomics and the utilization of metabolite bio-signatures, captured through machine learning, for detection of multiple early-stage cancers in adult males and females mammals.

### BACKGROUND OF THE INVENTION

Cancer is a leading cause of death worldwide, with the disease burden expanding in countries of all income levels due to growth and aging. In India, the estimated number of people living with the disease is around 2.25 million. Each year that swells to around 1.1 million and mortality rate is around 0.7 million/year. Risk of cancer development below 75 years of age in male and female are 9.81% and 9.42% respectively (http://cancerindia.org.in/cancer-statistics/). In the USA, 1700 people are expected to die from cancer each day (American Cancer Society. Economic impact of cancer. Page revised Jan. 3, 2018. Accessed Jul. 16, 2020. cancer.org/cancer/cancer-basics/economic-impact-of-cancer.html). The American Cancer Society (ACS) projections for 2020 include just over 1.8 million new cancer cases and around 0.6 million cancer deaths (Siegel R L, Miller K D, Jemal A. Cancer statistics, 2020. CA Cancer J Clin. 2020; 70(1):7-30. doi: 10.3322/caac.21590). Disease Control and Prevention (CDC) also provided similar estimations for new cancer cases and cancer deaths (CDC. Expected New Cancer Cases and Deaths in 2020. Page reviewed Aug. 16, 2018. Accessed Jul. 16, 2020. cdc.gov/cancer/dcpc/research/articles/cancer_2020.htm). Although the density of population in Europe represents only the tenth of the global population, yet one in four of all cancer diagnoses occur in this region. Breast, prostrate, lung and colorectal cancers represent over half of all cancer diagnoses in Europe (https://canceratlas.cancer.org/the-burden/europe/).

It is widely recognized that the most critical point for best prognosis is to identify cancer in its early stage as this could reduce death rates significantly in the long-term. Unfortunately, however, effective methodologies for early-stage cancer detection are either lacking, or not sufficiently sensitive, for many of the cancers that are of major public health relevance. For instance, there is yet no reliable method for detection of early-stage ovarian cancer, as well as for asymptomatic endometrial cancers. Similarly, for breast cancer, existing detection methods suffer from limitations that include either high cost, time consumption, and/or inadequate efficacy. For instance, Prostate Specific Antigen (PSA) test has been widely recommended for prostate cancer screening but this has test considered to be inaccurate, and is failing to identify eight of every 10 men aged under 60, who later have prostate cancer diagnosed (BMJ. 2003 Aug. 2; 327(7409): 249). In another instance, although mammography is the commonly recommended method for early detection, the relatively high false-positive and false-negative rates, particularly in patients with dense breasts, presents a problem. The efficacy of biomarker-based approaches, employing either DNA or protein markers, is also similarly compromised due either to poor penetration in the risk groups (DNA), or low circulating concentrations (proteins). In another instance, lung cancer screening with low-dose CT scans (LDCT) has shown to reduce the risk of dying from lung cancer. However, this screening platform is based on radiation therapy and its use over a prolonged period involving multiple sittings of the patient to control tumor propagation could further instigate some other health issues. In addition, this test shown to have a 12-14% false positive rate, which is high enough not to qualify as a screening test. In yet another instance, there are no widely recommended screening test available for patients with average risk of cancers (for example: Leukemia, Thyroid cancer, Melanoma, Kidney cancer, lymphoma, Pancreatic cancer, Liver and bile cancer). However, for some cancers such as Colorectal cancer, Gastric cancer and Head & Neck cancer there are currently no blood-based screening test available. These cancers (for example: Colorectal cancer, Gastric cancer and Head & Neck cancer) can only be early detected either using physical examination or a procedure that is invasive in nature. Finally, while screening strategies for early-stage cervical cancer do exist, their impact has been limited in less developed regions of the world where about 85% of new cases occur. Early detection of disease even accounting for lead time bias (which occurs when patients live longer due to earlier detection) and length bias (which occurs when early detection tests preferentially detect slower growing cancers, creating a false impression of longer survival), there remains a significant opportunity to reduce the burden of cancer with effective early detection. The true benefit of early detection is only realized if effective early treatment produces better results for patients and must not be confused with these biases (THE AMERICAN JOURNAL OF MANAGED CARE® Supplement VOL. 26, NO. 14 S293). These factors, therefore, emphasize the need for developing new methods that can detect early-stage cancers with a high degree of accuracy, and that are economical enough to be affordable across the economic spectrum. In this context, an integrated test that can simultaneously screen multiple cancers would provide a distinct advantage.

Metabolomics is an emerging field and is broadly defined as the comprehensive measurement of all metabolites and low-molecular-weight molecules in a biological specimen. Metabolomics affords profiling of much larger numbers of metabolites than are presently covered in standard clinical laboratory techniques. Hence it facilitates comprehensive coverage of biological processes and metabolic pathways. Consequently, it holds promise to serve as an essential objective lens in the molecular microscope for precision medicine. This is particularly relevant as metabolites have been described as proximal reporters of disease because their abundances in biological specimens are often directly related to pathogenic mechanisms.

The idea that the metabolite composition of biological fluids reflects the health of an individual has existed for a long time. Confidence in this supposition comes from experience with recent applications to find early metabolic indicators of disease in longitudinal cohorts, years before symptoms are clinically apparent for example, in pancreatic cancer, type 2 diabetes, cardiovascular disease, memory impairment, and many other conditions. Metabolomics studies have also inspired work revealing novel insights into relationships between diet and disease, such as observations linking elevated branched chain amino acids and obesity to insulin resistance. Such studies, therefore, provide strong support that metabolomics—coupled with multivariate statistical analysis—provides a relatively simple and efficient way to identify risk factors and/or biomarkers for disease.

Metabolomics is an especially relevant technique for cancer detection. Cancer cells have significantly altered metabolism and, therefore, the pattern of metabolites produced can yield a “signature” that is indicative of the cancer's presence or behavior. Importantly, and in contrast to gene expression profiling as a risk stratifier, this is a signal that originates directly or indirectly from micrometastatic disease, rather than one derived from features of the primary tumor. As a result, metabolome derived signatures provide a high-precision risk-stratifier for disease, with an accuracy that can far exceed those of methods based on DNA or protein markers. Untargeted metabolome profiles, however, are complex and multivariate in nature, and cannot be accurately analyzed by linear analytical methods. Such data, however, is readily amenable to the application of AI-based methodologies. By exploring non-linear variables in the data that correlate with defined clinical states, one can potentially extract metabolite signatures that are characteristic of a given disease state.

Metabolomics is now frequently used in oncology research, with particular emphasis on early diagnosis, monitoring, and prognosis of cancers. For example, several studies have exploited metabolomics analysis for both diagnosis and prognosis of breast cancer. Collectively, however, these studies have suffered from a variability in results, as well as limited accuracy. Similarly, the application of metabolomics for endometrial cancer resulted in the identification of metabolites that could predict the presence of cancer, tumor behavior, and also the pathological characteristics. These findings, however, await validation. A recent analysis identified metabolite signatures for cervical intraepithelial neoplasia and cervical cancer. The sample sizes though were relatively small and the discriminatory capacity of the test was sub-optimal. Metabolomic approaches for diagnosis of ovarian cancer has been recently reviewed. The inference was that while metabolomics offers significant new opportunities for ovarian cancer diagnosis, further work needed to be done.

U.S. Pat. No. 9,459,255 discloses amino acids that are useful in discriminating between breast cancer and breast cancer-free individuals. A multivariate discriminant was found, which included the concentrations of the identified amino acids as explanatory variables, that correlated significantly with the state of breast cancer. The sensitivity of the method, however, was only about 87% whereas the specificity was about 85%.

US 1992/5162504 discloses the use monoclonal antibodies that target the Prostate Specific Membrane Antigen (PSMA) that act as a cytogen's imaging agent for prostate cancer.

US 2011/0143444 discloses a method for evaluating female genital cancer, by using the amino acid concentrations in blood collected from subjects. This method evaluates the state of female genital cancer including at least one of cervical cancer, endometrial cancer, and ovarian cancer in the subject. The total number of subject samples tested, however, was small and the discriminatory power of the method was weak, ranging from 55% to 81% for the individual cancers.

US 2009/20120100558A1 demonstrated the onset of lung cancer by screening the biological fluid from patient. This invention relies on the presence of autoantibodies that are specific for one or more pre-diagnostic lung cancer indicator proteins such as LAMR1 and additionally or alternatively annexin I and/or 14-3-3-theta.

US 2017/0003291 is drawn to a method for diagnosing endometrial cancer by detecting, in a biological sample from a patient, variations in concentrations of specific lipids and some small metabolites. Using combined NMR and Mass spectrometry (MS) based metabolomics analysis, statistically significant changes were found in the serum of endometrial cancer patients in comparison with unaffected controls. However, despite that fact that two separate metabolome analysis techniques were employed, the resultant sensitivity and specificity of the method ranged only between 70% to 80%.

US 2017/0097355 describes methods for measuring metabolic changes useful in the differentiation between ovarian cancer and benign ovarian tumor. Two independent LC-MS-based metabolomics platforms, including a global lipidomics approach, were used to screen for differentially abundant plasma metabolites between cases with serous ovarian carcinoma and controls with benign serous ovarian tumor. While the combination of small molecule with lipidome profiling yielded test with good sensitivity (95%), the specificity however was less than 50%. This limits the utility of the test for patient screening.

Thus, it is clear from all of these studies that better methods, with higher fidelity, are required for early-stage diagnosis of multiple cancers simultaneously. Furthermore, it is also evident that screening for early-stage cancers would greatly benefit the survival chances of patients. Thus, it is eminently desirable to develop a single non-invasive test that can efficiently screen for multiple cancers simultaneously, using small volumes of biological fluids.

### OBJECT OF THE INVENTION

The objective of the invention is to revolutionize the early-stage detection of multiple cancers in both adult males and females. The innovation focuses on leveraging clinical metabolomics and machine learning to capture metabolite bio-signatures, aiming for accurate and comprehensive cancer detection. With the global significance of cancer as a leading cause of mortality in mind, the invention aims to address the burden of the disease on a global scale, targeting countries with varying income levels, including India and the USA and various other countries, to improve early detection rates.

Yet another object of the invention is overcoming the limitations associated with existing early-stage cancer detection methods, particularly for cancers such as ovarian, endometrial, and breast cancer. The invention address issues related to accuracy, cost, time consumption, and efficacy in current detection methods. To achieve this, a comprehensive metabolomics approach is employed, utilizing metabolomics as a high-precision risk stratifier and gaining essential insights into metabolic pathways related to cancer.

Another object of the invention is the integration of advanced Artificial Intelligence (AI) and Machine Learning (ML) processes plays a crucial role in enhancing the accuracy of cancer detection. The emphasis is on AI/ML's potential to analyze complex and multivariate metabolome profiles for efficient disease identification. The invention strives to develop a non-invasive test capable of simultaneously screening multiple cancers through a single analysis, minimizing the need for invasive procedures and providing an efficient screening approach for various cancer types. The core technology employed for resolving metabolites in biological fluid samples is Liquid Chromatography-Mass Spectrometry (LC-MS). The focus is on optimizing LC-MS to ensure accurate measurement of masses for metabolite ions and obtaining ion spectra. Robust quality control processes are established to identify and rectify errors in the detection of multiple cancers. This includes the implementation of a sequential neural network model, monitoring critical ions, and assessing matrix occupancy to enhance data accuracy.

One more object of the invention involves the creation of specific AI models, namely the Cancer Detection AI (CDAI) Model for distinguishing cancerous samples from normal ones and the Tissue Of Origin Identification (TOOAI) Model for identifying specific cancer types based on tissue origin using multiclass classification. With a vision of global impact and affordability, the invention aims to contribute to reducing cancer-related mortality across diverse populations, prioritizing accessibility across various economic spectra. Validation and accuracy assessment are paramount, with an emphasis on validating AI models through logistic regression, class balancing, and optimization processes. Systematic evaluation using training and test datasets ensures the accuracy of the developed methodologies. Ultimately, the invention strives to revolutionize early cancer detection by combining advanced technologies, comprehensive metabolomics, and AI/ML methodologies, aiming to make a significant and impactful contribution to the field of precision medicine.

### SUMMARY OF THE INVENTION

The present invention relates to a system and method for the simultaneous early detection of multiple cancers through a single analysis. The system involves a Liquid Chromatography-Mass Spectrometry (LC-MS) device, processors for data analysis and quality control, and AI/ML processes for cancer detection. The LC-MS device resolves metabolites in biological fluid samples, and the system aligns and normalizes mass data while minimizing errors. Quality control processes include building a neural network, monitoring critical ions, and assessing matrix occupancy. AI/ML processes create a Cancer Detection AI (CDAI) Model and a Tissue Of Origin Identification (TOOAI) Model to differentiate cancerous and normal samples and identify specific cancer types. The LC-MS device also includes sample collection, extraction, and reconstitution components. The method involves analyzing metabolite ions, applying quality control, and employing AI/ML processes for cancer detection and tissue origin identification. The AI models are created through logistic regression and multiclass classification. The accuracy of these models is evaluated using training and test datasets. The system aims to revolutionize early cancer detection through comprehensive analysis and advanced machine learning techniques.

Another embodiment of the present invention provides a system for simultaneous detection of multiple cancers at early stages in a single analysis, the system comprising: at least one Liquid Chromatography (LC) device with a mass spectrometer (MS) (abbreviated, herein after, as LC-MS) for analysing and measuring masses for metabolite ions in one or more resultant reconstituted metabolites, using the LC-MS technique, the one or more resultant reconstituted metabolites being obtained after reconstituting of one or more dried metabolite extracts extracted from one or more biological fluid samples; at least one processor/computing device to align the masses obtained from metabolome profile of the metabolite ions in the one or more resultant reconstituted metabolites, and to minimize errors that may be generated in measurement of the masses for the metabolite ions; at least one processor/computing device applying one or more quality control processes on the aligned and normalized dataset to identify any errors that may have occurred in the detection of multiple cancers in the biological fluid samples, the at least one processor/computing device executing quality control processes is configured to execute at least one or more of the following steps (a), (b) and (c):
(a) build a sequential neural network model to detect the errors based on variations in chromatogram profiles of faulty sample extraction, or due to an error in the mass spectrometry;
(b) monitor for the presence of at least 6 out of 9 critical ions with m/z values ranging from 100 to 800, wherein the presence of 6 or more of the 9 critical ions in the chromatogram of a sample is taken as the threshold criteria for passing a quality control step; and
(c) assess a matrix occupancy that includes calculating a threshold for minimum matrix occupancy with multiple runs of poor-quality samples, or with improper mass spectrometry run conditions; and

at least one processor executing one or more AI/ML process on the measured metabolite ions: to create a first AI Model (Cancer Detection AI (CDAI) Model) for identifying and differentiating diseased cancerous samples from non-diseased normal samples, where the measured metabolite ions is randomly divided into a training dataset and a test dataset, to create a second AI Model (a Tissue Of Origin Identification (TOOAI Model)) to further identify and differentiate individual diseased cancerous sample from other diseased cancerous samples and the non-diseased normal samples, and to apply the TOOAI Model over cancer-positive samples determined by the CDAI Model to identify and differentiate individual diseased cancerous sample from the other diseased cancerous samples and the non-diseased normal samples by obtaining scores assigned to each of the individual diseased cancerous sample, where one score for each cancer type is defined by its tissue of origin, and denotes the probability of the sample belonging to the respective cancer type.

Yet another embodiment of the present invention provides a system wherein the LC-MS device is configured to: resolve the one or more resultant reconstituted metabolites by an Ultra High-Performance Liquid Chromatography using the LC device; obtain ion spectra of the one or more resultant reconstituted metabolites through the MS device; and measure masses for metabolite ions present in the ion spectra, of the one or more resultant reconstituted metabolites, based on their mass-to-charge ratio or m/z through the MS device.

One more embodiment of the present invention provides a system wherein to create at the first AI Model (Cancer Detection AI (CDAI) Model), the at least one processor/computing device is configured to: apply a logistic regression function by executing the AI/ML processes on the training dataset of the metabolite ions to find a functional mapping between dependent/target variable and independent variable that separates diseased cancerous samples from non-diseased normal samples; configure one or more class balancing parameters for the target variable to balance the imbalance of classes in the training dataset; apply an optimization processes to handle complexity of data, thereby creating the first AI Model from the training dataset, the first AI model is the Cancer Detection AI (CDAI) Model; and apply the CDAI Model on the test dataset of the metabolite ions for identifying and differentiating the diseased cancerous samples from the non-diseased normal samples based on the function separating diseased cancerous samples from non-diseased normal samples. Further, to create the second AI model (Tissue Of Origin Identification (TOOAI Model)), the at least one processor/computing device is configured to: configure a classifier multiclass classification model using the training dataset, and thereby creating the second AI Model from the training dataset, the second AI model is a Tissue Of Origin Identification (TOOAI Model), and the classifier multiclass classification model includes at least one or more of a support vector machine, a logistic one versus rest, or a stochastic gradient descent processes; and apply the TOOAI Model over cancer-positive samples determined by the CDAI Model to identify and differentiate individual diseased cancerous sample from the other diseased cancerous samples and the non-diseased normal samples by obtaining scores assigned to each of the individual diseased cancerous sample, where one score for each cancer type is defined by its tissue of origin, and denotes the probability of the sample belonging to the respective cancer type. The system further comprises: at least one sample collecting device for collecting the one or more biological fluid samples from one or more biological mammals; at least one precipitating device for extraction of the one or more metabolite extracts from the one or more biological fluid samples by precipitation of protein present in the biological fluid samples with chilled alcohol including at least methanol; at least one phase separation device for drying the one or more metabolite extracts extracted from the at least one precipitating device; and at least one device for reconstituting the one or more dried metabolite extracts in aqueous solutions in a mobile phase.

One more embodiment of the present invention provides a system wherein the at least one processor/computing device to create at the first AI Model (Cancer Detection AI (CDAT) is further configured to find a score for each sample in the training set, using a resulting trained model/processes from the CDAI model, and to evaluate the test set to determine the accuracy applying the trained CDAI model.

One more embodiment of the present invention provides a system wherein the at least one processor/computing device executing the AI/ML processes to create the second AI model (Tissue Of Origin Identification (TOOAI Model)) is further configured to determine accuracy of the multiclass classification model in the TOOAI Model in differentiating individual diseased cancerous sample from the other diseased cancerous samples and the non-diseased normal samples, wherein in determining the accuracy, the at least one processor/computing device executing the AI/ML processes is further configured to:
obtain and provide probability scores for each cancer subclass for every given sample of an individual diseased cancerous sample; wherein the probability scores of one individual diseased cancerous sample differentiates the probability scores for other diseased cancerous samples and other diseased cancerous sample;
taking the top two scoring cancer subclasses were as the model result and matching with the true labels; and
building a final confusion matrix based on double class accuracy of the model.

Yet another embodiment of the present invention provides a method for simultaneous detection of multiple cancers at early stages in a single analysis, the method comprising: analysing and measuring masses for metabolite ions in one or more resultant reconstituted metabolites, using a LC-MS technique, the one or more resultant reconstituted metabolites being obtained after reconstituting of one or more dried metabolite extracts extracted from one or more biological fluid samples; aligning the masses obtained from metabolome profile of the metabolite ions in the one or more resultant reconstituted metabolites, and minimizing errors that may be generated in measurement of the masses for the metabolite ions; applying one or more quality control processes on the aligned and normalized dataset to identify any errors that may have occurred in the detection of multiple cancers, the one or more quality control processes is configured to execute at least one or more of the following steps (a), (b) and (c):
(a) build a sequential neural network model to detect the errors based on variations in chromatogram profiles of faulty sample extraction, or due to an error in the mass spectrometry;
(b) monitor for the presence of at least 6 out of 9 critical ions with m/z values ranging from 100 to 800, wherein the presence of 6 or more of the 9 critical ions in the chromatogram of a sample is taken as the threshold criteria for passing a quality control step; and
(c) assess a matrix occupancy that includes calculating a threshold for minimum matrix occupancy with multiple runs of poor-quality samples, or with improper mass spectrometry run conditions; and

executing one or more AI/NIL process on the measured metabolite ions: to create at a first AI Model (Cancer Detection AI (CDAI) Model) for identifying and differentiating diseased cancerous samples from non-diseased normal samples, where the measured metabolite ions is randomly divided into a training dataset and a test dataset, to create at a second AI Model (a Tissue Of Origin Identification (TOOAI Model)) to further identify and differentiate individual diseased cancerous sample from other diseased cancerous samples and the non-diseased normal samples, and to apply the TOOAI Model over cancer-positive samples determined by the CDAI Model to identify and differentiate individual diseased cancerous sample from the other diseased cancerous samples and the non-diseased normal samples by obtaining scores assigned to each of the individual diseased cancerous sample, where one score for each cancer type is defined by its tissue of origin, and denotes the probability of the sample belonging to the respective cancer type.

One more embodiment of the present invention provides a method wherein the LC-MS technique further includes: resolving the one or more resultant reconstituted metabolites by an Ultra High-Performance Liquid Chromatography using the LC device; obtaining ion spectra of the one or more resultant reconstituted metabolites through a MS device; and measuring masses for metabolite ions present in the ion spectra, of the one or more resultant reconstituted metabolites, based on their mass-to-charge ratio or m/z through the MS device.

Yet another embodiment of the present invention provides a method wherein to create at the first AI Model (Cancer Detection AI (CDAI) Model), the one or more AI/ML processes are further executed to: apply a logistic regression function on the training dataset of the metabolite ions to find a functional mapping between dependent/target variable and independent variable that separates diseased cancerous samples from non-diseased normal samples; configure one or more class balancing parameters for the target variable to balance the imbalance of classes in the training dataset; apply an optimization process to handle complexity of data, thereby creating the first AI Model from the training dataset, the first AI model is the Cancer Detection AI (CDAI) Model; and apply the CDAI Model on the test dataset of the metabolite ions for identifying and differentiating the diseased cancerous samples from the non-diseased normal samples based on the function separating diseased cancerous samples from non-diseased normal samples.

One more embodiment of the present invention provides a method, wherein to create the second AI model (Tissue Of Origin Identification (TOOAI Model)), the one or more AI/ML processes are further executed to: configure a classifier multiclass classification model using the training dataset, and thereby creating the second AI Model from the training dataset, the second AI model is a Tissue Of Origin Identification (TOOAI Model), and the classifier multiclass classification model includes at least one or more of a support vector machine, a logistic one versus rest, or a stochastic gradient descent processes; and apply the TOOAI Model over cancer-positive samples determined by the CDAI Model to identify and differentiate individual diseased cancerous sample from the other diseased cancerous samples and the non-diseased normal samples by obtaining scores assigned to each of the individual diseased cancerous sample, where one score for each cancer type is defined by its tissue of origin, and denotes the probability of the sample belonging to the respective cancer type.

Other embodiment of the present invention provides a method, wherein the method further comprises: collecting the one or more biological fluid samples from one or more biological mammals; extraction of the one or more metabolite extracts from the one or more biological fluid samples by precipitation of protein present in the biological fluid samples with chilled alcohol including at least methanol; drying the one or more metabolite extracts extracted from the at least one precipitating device; and reconstituting the one or more dried metabolite extracts in aqueous solutions in a mobile phase. Further to create at the first AI Model (Cancer Detection AI (CDAI), the one or more AI/ML processes are further executed to find a score for each sample in the training set, using a resulting trained model/processes from the CDAI model, and to evaluate the test set to determine the accuracy applying the trained CDAI model. Furthermore, to create the second AI model (Tissue Of Origin Identification (TOOAI Model)), the one or more AI/ML processes are further executed to determine accuracy of the multiclass classification model in the TOOAI Model in differentiating individual diseased cancerous sample from the other diseased cancerous samples and the non-diseased normal samples, wherein in determining the accuracy, the one or more AI/ML processes are further executed to: obtain and provide probability scores for each cancer subclass for every given sample of an individual diseased cancerous sample; wherein the probability scores of one individual diseased cancerous sample differentiates the probability scores for other diseased cancerous samples and other diseased cancerous sample; taking the top two scoring cancer subclasses were as the model result and matching with the true labels; and building a final confusion matrix based on double class accuracy of the model.

One more embodiment of the present invention provides a system wherein the metabolome profile of the metabolite ions is generated using an automatic platform which includes at least the compound discoverer.

### BRIEF DESCRIPTION OF THE FIGURES

FIG. 1 depicts Workflow of the overall steps involved in cancer detection.

FIG. 2 depicts schematic depiction of the overall processes under study. This process includes the sample preparation which, in principle relies on protein precipitation to extract the metabolites. The extracted metabolites were phase separated, and the extract was dried under a vacuum. Finally, UHPLC-IRMS was employed to separate the metabolites based on their retention ability on an Acquity UPLC HSS T3 column from Waters (1.8 micron, dimensions—2.1×100 mm, Part No. 186003539). Prior to AI/ML workflow, samples were subjected to quality check modules QC, QC2 and QC3 for the authentication of sample extraction and chromatogram. These separated features were then subjected to AI/ML based analysis for pattern recognition.

FIG. 3 depicts Age-wise distribution of samples among healthy and cancer individuals. A total number of 8971 cancer serum samples of 33 mentioned cancers were collected with 3914 samples represented the normal control set.

FIG. 4 depicts Number of metabolites present across the samples of normal control and the 33 mentioned cancer. The cancers and normal controls are grouped based on age interval i.e <40 years, 40-60 years and >60 years.

FIG. 5 depicts mass and Retention time index for each ion box. The figure depicts the Mass error for each metabolite (FIG. 3A) and retention time variation (FIG. 3B) for each Mass box/metabolite.

FIG. 6 depicts quality checks for sample verification. These are QC1, QC2 and QC3. QC1 determine the spectra quality that fits the approved criteria of chromatogram. QC2 employed to find the >5 critical masses out of 9 designated critical masses in the spectrum. While, QC3 approved the 0.2 matrix occupancy in the samples as correct chromatogram.

FIG. 7 depicts PLS DA Plot of the matrix of samples and metabolites versus metabolite intensity showing the clear separation of samples based on their clinical information.

FIG. 8 depicts AI workflow: Multi cancer detection platform/Tissue of origin detection. The workflow depicts the three major compartments common to the layer model left to right data processing, Train test split, model building testing.

FIG. 9 depicts Testing the trained Layer I model for Cancer versus Normal and Disease Controls showing clear separation of Cancers versus Controls based on model scores. The y score of each of the 33 cancers are shown separately. The resulting confusion matrix on applying the threshold of 0 shows high accuracy, sensitivity, and specificity.

FIG. 10 depicts Testing the Multiclass Trained Model Layer2 model for Tissue identification. The resultant confusion matrix is generated after applying double class prediction from the layer 2 model i.e., Tissue identification for cancer positive sample.

FIG. 11 depicts Coefficient/weights of each metabolite involved in the signature of Cancer to be differentiated from the normal controls.

Table 1: shows the distribution of the 33 cancer and normal control samples on the basis of parameters like Age interval, BMI, Ethnicity, Cancer stage.

### DETAILED DESCRIPTION OF THE INVENTION

Although specific terms are used in the following description for the sake of clarity, these terms are intended to refer only to the particular structure of the invention selected for illustration in the drawings and are not intended to define or limit the scope of the invention.

References in the specification to “one embodiment” or “an embodiment” member that a particular feature, structure, characteristics, or function described in connection with the embodiment is included in at least one embodiment of the invention. The appearances of the phrase “in one embodiment” in various places in the specification are not necessarily all referring to the same embodiment.

The present invention discloses embodiments that enable simultaneous screening for multiple cancers such as endometrial cancer, breast cancer, cervical cancer, lung cancer, prostate cancer and ovarian cancer but not limited to the name specified here, in a single analysis. The present invention related to a system and a method that may integrate global metabolome profiling with machine learning powered data analysis, to capture the disease-specific signatures.

In an embodiment, the invention may provide an integrated method for the simultaneous detection of multiple cancers. This method may further elaborate the process of untargeted metabolomics for detecting and measuring metabolic changes that are not only useful in the broad differentiation between cancer and healthy individual but also effectively, and simultaneously, distinguish each individual cancer from normal controls as well as the other cancers.

Although, the detailed description herein explains and relates to the multiple cancers, which are endometrial cancer, breast cancer, cervical cancer, ovarian cancer, lung cancer, leukemia, thyroid cancer, melanoma, colorectal cancer, kidney cancer, lymphoma, pancreatic cancer, liver & bile duct cancer, gastric cancer, larynx cancer, pharynx cancer, oral cancer, esophageal cancer, prostate cancer, bladder cancer, brain and CNS cancer, multiple myeloma, anus cancer, testicular cancer, vulva cancer, penile cancer, vagina cancer, gallbladder cancer, sarcoma cancer, germ cell tumor, squamous cell carcinoma and unknown primary, but the method explained here may not be restricted in detection these cancers only, and may be applied on segregation and detection of other cancer in a biological mammal specimen from normal controls.

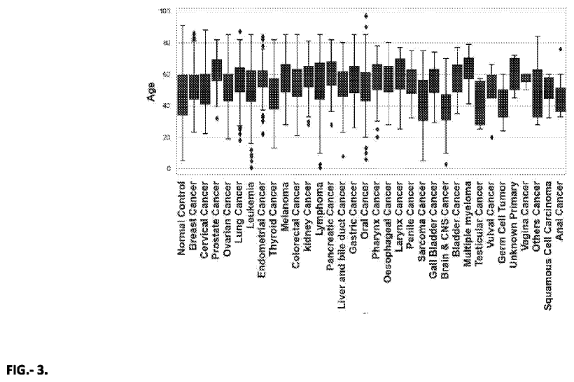

As described in some of the examples below, a Liquid Chromatography with mass spectrometry (abbreviated as LC-MS) based untargeted metabolomics approach may be used to screen differentially abundant serum metabolites from control cases (normal controls and disease controls) and test cases (i.e. either endometrial cancer, breast cancer, cervical cancer, ovarian cancer, lung cancer, leukemia, thyroid cancer, melanoma, colorectal cancer, kidney cancer, lymphoma, pancreatic cancer, liver & bile duct cancer, gastric cancer, larynx cancer, pharynx cancer, oral cancer, esophageal cancer, prostate cancer, bladder cancer, brain and CNS cancer, multiple myeloma, anus cancer, testicular cancer, vulva cancer, penile cancer, vagina cancer, gallbladder cancer, sarcoma cancer, germ cell tumor, squamous cell carcinoma and unknown primary). In a particular example study, which is conducted and presented herein, a total of 8971 serum samples were collected from participants. Among them, 3914 were designated as normal controls, while 5057 were selected as test cases. In the test cases, the distribution of cancers serum samples was 445, 652, 458, 488, 307, 157, 169, 151, 296, 136, 97, 134, 147, 279, 20, 52, 566, 143, 122, 32, 42, 18, 9, 4, 5, 18, 2, 35, 45, 14, 8, and 6 as endometrial cancer, breast cancer, cervical cancer, ovarian cancer, lung cancer, leukemia, thyroid cancer, melanoma, colorectal cancer, kidney cancer, lymphoma, pancreatic cancer, liver & bile duct cancer, gastric cancer, larynx cancer, pharynx cancer, oral cancer, esophageal cancer, prostate cancer, bladder cancer, brain and CNS cancer, multiple myeloma, anus cancer, testicular cancer, vulva cancer, penile cancer, vagina cancer, gallbladder cancer, sarcoma cancer, germ cell tumor, squamous cell carcinoma and unknown primary, respectively, while “other” cancer has total of 16 samples distribution TABLE-1 (shown in FIG. 3). The potential utility of derived metabolite profiles to discriminate between cases and controls, in the example study of the present invention and, was investigated through construction and evaluation of multivariate classification matrix.

Before describing exemplary embodiments in greater detail, the following definitions are set forth to illustrate and define the meaning and scope of the terms used in the description. Unless defined otherwise, all technical and scientific terms used here in have the same meaning as commonly understood by one of ordinary skill in the art to which this invention belongs. Singleton, et al., DICTIONARY OF MICROBIOLOGY AND MOLECULAR BIOLOG, 2D ED., John Wiley and Sons, New York (1994), and Hale & Markham, THE HARPER COLLINS DICTIONARY OF BIOLOGY, Harper Perennial, N.Y. (1991) provide one of skill with the general meaning of many of the terms used herein. Still, certain terms are defined below for the sake of clarity and ease of reference.

It must be noted that as used herein and in the appended claims, the singular forms “a”, “an”, and “the” include plural referents unless the context clearly dictates otherwise. For example, the term “a sample” refers to one or more samples, i.e., a single sample and multiple samples. As such, this statement is intended to serve as antecedent basis for use of such exclusive terminology as “solely” “only” and the like in connection with their citation of claim laments, or use of a “negative” limitation.

The term “sample” as used herein relates to a material or mixture of materials, typically, although not necessarily, in liquid form, containing one or more analytes of interest. In one embodiment, the term as used in its broadest sense, refers to any mammalian material containing cells or producing cellular metabolites, such as, for example, tissue or fluid isolated from an individual (including without limitation plasma, serum, cerebrospinal fluid, lymph, tears, saliva and tissue sections) or from in vitro cell culture constituents, as well as samples from the environment. The term “sample” may also refer to a “biological sample”. As used herein, the term “a biological sample” refers to a whole organism or a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A “biological sample” can also refer to a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genito-urinary tracts, tears, saliva, milk, blood cells, tumors, organs. In certain embodiments, the sample has been removed from an animal. Biological samples of the invention include cells.

Metabolite profile as used in the invention should be understood to be any defined set of values of quantitative results for metabolites that can be used for comparison to reference values or profiles derived from another sample or a group of samples. For instance, a metabolite profile of a sample from a diseased patient might be significantly different from a metabolite profile of a sample from a similarly matched healthy patient. Metabolites can be, but not limited to, amino acids, peptides, acylcarnitines, monosaccharides, lipids and phospholipids, prostaglandins, steroids, bile acids and glycol and phospholipids can be detected and/or quantified.

As used herein, untargeted metabolomics studies are characterized by the simultaneous measurement of many metabolites from biological samples. This strategy, known as top-down strategy, avoids the need for a prior specific hypothesis on a particular set of metabolites and, instead, analyses the global metabolomic profile. Consequently, these studies are characterized by the generation of large amounts of data. This data is not only characterized by its volume but also by its complexity and, therefore, there is a need for high performance bioinformatic tools.

As used herein, the term chromatography refers to a process in which a chemical mixture carried by a liquid or gas is separated into components as a result of differential distribution of the chemical entities as they flow around or over a stationary liquid or solid phase.

As used herein, the term high performance liquid chromatography or HPLC (also sometimes known as high pressure liquid chromatography) refers to liquid chromatography in which the degree of separation is increased by forcing the mobile phase under pressure through a stationary phase, typically a densely packed column. As used herein the term ultra-high performance liquid chromatography or UPLC or UHPLC (sometimes known as ultra-high pressure liquid chromatography) refers to HPLC which occurs at much higher pressures than traditional HPLC techniques.

As used herein, the term sample injection refers to introducing an aliquot of a single sample into an analytical instrument, for example a mass spectrometer. This introduction may occur directly or indirectly. An indirect sample injection may be accomplished, for example, by injecting an aliquot of a sample into a HPLC or UPLC analytical column that is connected to a mass spectrometer in an on-line fashion.

As used herein, the term mass spectrometry or MS refers to an analytical technique to identify compounds by their mass. MS refers to methods of filtering, detecting and measuring ions based on their mass-to-charge ratio or m/z.

As used herein, the term operating in positive ion mode refers to those mass spectrometry methods where positive ions are generated and detected.

As discussed herein, the term electron ionization or EI refers to methods in which an analyte of interest in a gaseous or vapor phase interacts with a flow of electrons. Impact of the electrons with the analyte produces analyte ions, which may then be subjected to a mass spectrometry technique.

As used herein, the term electrospray ionization or ESI refers to methods in which a solution is passed along a short length of capillary tube, to the end of which is applied a high positive or negative electric potential. Solution reaching the end of the tube is vaporized (nebulized) into a jet or spray of very small droplets of solution in solvent vapor. This mist of droplets flows through an evaporation chamber, which is heated slightly to prevent condensation and to evaporate solvent. As the droplets get smaller, the electrical surface charge density increases until such time that the natural repulsion between like charges causes ions as well as neutral molecules to be released.

As used herein, data processing involves typically the data reduction step called filtering. Noise filters reduce the data based on a calculated noise threshold. In this respect, data below a certain signal to noise ratio is filtered. Content based filtering of the results leverages. For example, disease specific knowledge to concentrate on relevant metabolite aspects of the disease under investigation.

After pre-processed data derived from mass spectrometry analysis has been technical validated, statistical analysis can proceed. Depending on the design of a metabolite profiling study, a sample or several samples derived from healthy controls and patients are compared to reveal differences, i.e. biomarkers that can be utilized to characterize a disease at the molecular level. In another embodiment, samples are derived from patients participating in a clinical trial, where a novel drug compound is under investigation and compared to an approved drug.

Artificial intelligence in its core, the new technical discipline that researches and develops theories, methods, technologies, and application system for simulating the extension and expansion of human intelligence. The use of AI in research likely to perform some complex tasks that require human cognitive ability. The major core concept of AI is machine learning and deep learning. However, machine learning is the art of study of algorithms that learn from examples and experiences. Additionally, machine learning is based on the idea that there exist some patterns in the data that were identified and used for future predictions. While deep learning uses different layers to learn from the data. The depth of the model is represented by the number of layers in the model. In deep learning, the learning phase is done through a neural network. A neural network is an architecture where the layers are stacked on top of each other.

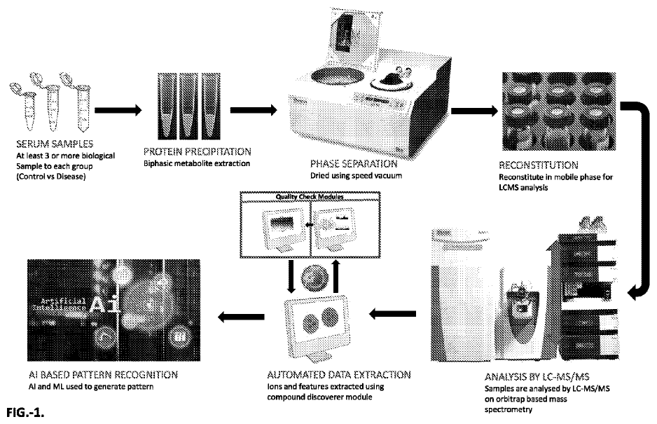

Referring to FIG. 1 that illustrates a schematic representation of a system for implementing metabolomics process for differentiating the cancer types (for example, endometrial cancer, breast cancer, cervical cancer, ovarian cancer, lung cancer, leukemia, thyroid cancer, melanoma, colorectal cancer, kidney cancer, lymphoma, pancreatic cancer, liver & bile duct cancer, gastric cancer, Larynx cancer, pharynx cancer, oral cancer, esophageal cancer, prostate cancer, bladder cancer, brain and CNS cancer, multiple myeloma, anus cancer, testicular cancer, vulva cancer, penile cancer, vagina cancer, gallbladder cancer, sarcoma cancer, germ cell tumor, squamous cell carcinoma and unknown primary and additional cancer grouped under ‘other’ category) from the normal controls and further distinguish cancer type among the group of cancer, and also the implementation of QCs to improve the accuracy of the prediction, in accordance with an embodiment of the present invention. The FIG. 1 shows a metabolomics system 100 that may comprise at least one or more components performing one or more functions from the following:
1. At least one sample collecting device 102 for collecting one or more biological fluid samples from one or more biological mammals;
2. At least one precipitating device 104 for extraction of one or more metabolite from the one or more biological fluid samples by precipitation of protein present in the biological fluid with chilled alcohol including at least methanol;
3. At least one vacuum dryer device 106 for drying the one or more metabolite extracts from the at least one precipitating device;
4. At least one device 108 for reconstituting the one or more dried metabolite extracts in aqueous solutions;
5. At least one liquid Chromatography (LC) device 110 with a mass spectrometer (MS) (abbreviated, herein after, as LC-MS) for analysing one or more resultant reconstituted metabolites;
6. At least one computing device 112 to align the masses obtained from the metabolome profile, generated using automatic platform i.e, compound discoverer software that extract data for metabolite ions and their related features;
7. At least one device to minimize the errors that may be generated in measurement of the masses for the ions;
8. At least one computing device 114 to subject the aligned and normalised ion spectra to three quality controls i.e a) Faulty chromatogram profile identifies using sequential neural network model. That ensures to eliminate any errors that are due to either faulty sample extraction or due to an error in the mass spectrometry
   b) Monitoring the presence of at least 6 out of 9 critical ions with m/z values ranging from 100 to 800. That confirms the high likelihood of accurate identification of cancer samples
   c) Matrix occupancy determines the percentage of features matches with the matrix size. That certifies the detection robustness and accuracy; and
9. At least one computing device 116 that may execute one or more AI/N algorithms for AI based pattern recognition for finally identifying, differentiating and presenting the cancer samples from the normal control samples and further to identify, differentiate and present individual cancer samples within the identified cancer samples. Thus, not only the detection and differentiation between the cancer and healthy individual can be achieved from the present system 100, but also effectively, and simultaneously, the present system 100 may distinguish each individual cancer from normal controls as well as the other cancer samples.

It should be again noted that FIGS. 1-11 will be explained taking examples and hence, should not be considered as limiting to those specific examples only. For example, the FIGS. 1-11 are described, herein, considering a sample size of 8971 taken from both male and female adult volunteers.

In an embodiment, the present system 100 may be implemented to distinguish multiple cancers from normal controls and, in addition, to subsequently differentiate between endometrial cancer, breast cancer, cervical cancer, ovarian cancer, lung cancer, leukemia, thyroid cancer, melanoma, colorectal cancer, kidney cancer, lymphoma, pancreatic cancer, liver & bile duct cancer, gastric cancer, head and neck cancer, esophageal cancer, and prostate cancer.

A number of samples are acquired from adult volunteers who are either free of any cancer (normal control) (n=3914), or have endometrial cancer (n=445), breast cancer (n=652), cervical cancer (n=458), ovarian cancer (n=488), lung cancer (n=307), leukemia (n=157), thyroid cancer (n=169), melanoma (n=151), colorectal cancer (n=296), kidney cancer (n=136), lymphoma (n=97), pancreatic cancer (n=134), liver & bile duct cancer (n=147), gastric cancer (n=279), larynx cancer (n=20), pharynx cancer (n=52), oral cancer (n=566), esophageal cancer (n=143), prostate cancer (n=122), bladder (n=32), brain and CNS cancer (n=42), multiple myeloma (n=18), anus cancer (n=9), testicular cancer (n=4), vulva cancer (n=5), penile cancer (n=18), vagina cancer (n=2), gallbladder cancer (n=35), sarcoma cancer (n=45), germ cell tumor (n=14), squamous cell carcinoma (n=8), unknown primary (n=6) and other cancers (n=16) TABLE-1. The samples are collected and stored in the sample collecting device 102. In an embodiment, the sample collecting device 102 may be a test tube.

Further, the present system 100 may include a metabolite extraction which may be achieved by precipitating serum proteins with chilled methanol, according to an embodiment. Thus, the precipitation device 104 may be used here in order to extract metabolite from the samples collected by precipitating serum proteins with chilled methanol. In an embodiment, the precipitation device 104 may be a test tube.

The supernatant may be collected as the metabolite extract and may further be dried before use. For the process of drying, in an embodiment, the phase separation device or a Vacuum dryer device 106 may be used that may dry the metabolite extract using speed vacuum.

Further, in an embodiment, the dried extract may be reconstituted in an aqueous solution in a mobile phase using a reconstituting device 108. Thereafter, the ion spectrum of the resultant samples, derived from the reconstitution phase, may be generated by LCMS, where samples may be first resolved by Liquid Chromatography (abbreviated as LC) with mass spectrometry (MS) (abbreviated, herein after, as LCMS) device 110. Using the device 110, ions in the metabolite extraction may be measured, the masses for the ions may be measured based on their mass-to-charge ratio or m/z FIG. 1.

Thereafter, the features of the ion spectra accumulated in metabolic profile may be extracted using compound discoverer software 112 (for example of compound discoverer software Thermo Fisher Scientific). The masses obtained for the ions in the metabolome profile, using the LCMS device 110, may be aligned across all the samples. This may be done to enable comparison of the peak intensity of each ion across all the samples. For example: a pool of known internal standard used for RT alignment with ±0.02 mins of error window, followed by peak picking and identification of metabolites.

The present system 100 may also include functions for minimizing the errors that be generated in measurement of the masses for the ions. To normalize for unavoidable, but minor, variations in mass (m/z), a sophisticated approach of using parts per million (ppm) error-based approach may be used, according to an embodiment. In another embodiment, briefly, a modified virtual lock mass-based approach may also be used. This is based on the principle that mass errors are known to increase with mass. This modified virtual lock mass-based approach may be used and adapted according to the datasets in examples of the invention. This may be done by combining the traditional virtual lock mass approach with metabolite identification from the Human Metabolome Database (HMDB). Specifically, the virtual lock mass boxes may be defined using the masses of metabolites identified by HMDB database search across multiple samples. Subsequently, the metabolite ions may be filtered based on the frequency of presence in samples may be used for metabolite ions filtering; meaning ions present in greater than 15% of samples may be used in subsequent analysis.

To improve the overall accuracy of the prediction using one or more AI/ML algorithms, a system 114 was introduced that was comprised of QC1, QC2 and QC3 steps. System 114 was applied on the aligned and normalized dataset to establish the confirmation of samples processed as per the optimized protocol. System 114 identifies any errors that may have occurred during sample processing in any of the steps of system 100. Implementation of system 114 is critical for the improvement of accuracy at the levels of both CDAI and TOOAI predictions.

Thereafter, on the obtained, measured, aligned, corrected and featured metabolite ions, which are measured and aligned as explained above, AI/ML models are applied for statistical analysis of the samples. The computing device 116 that may be able to execute one or more AI/ML algorithms for applying the AI/ML models for statistical analysis of the samples.

By executing the one or more AI/ML algorithms, using one or more processors, at the computing device 116, one or more first AI/ML models may be generated to first distinguish the cancer samples (endometrial cancer, breast cancer, cervical cancer, ovarian cancer, lung cancer, leukemia, thyroid cancer, melanoma, colorectal cancer, kidney cancer, lymphoma, pancreatic cancer, liver & bile duct cancer, gastric cancer, Larynx cancer, pharynx cancer, oral cancer, esophageal cancer, prostate cancer, bladder cancer, brain and CNS cancer, multiple myeloma, anus cancer, testicular cancer, vulva cancer, penile cancer, vagina cancer, gallbladder cancer, sarcoma cancer, germ cell tumor, squamous cell carcinoma and unknown primary, and ‘other’ cancers) from the normal controls. Subsequently, in another embodiment one or more additional AI/ML algorithms may be executed by using one or more processors, at the computing device 116, to further distinguish between the individual cancers (e.g., lung cancer from the remaining 18 cancers) TABLE-2.

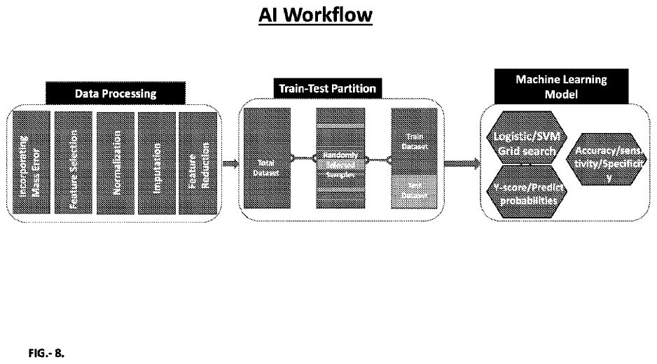

While generating the AI/Models, the computing device 116 may follow one or more of the following steps FIG. 8:
i. While developing the AI model, a functional mapping is established in between dependent/target variable and independent variable learning in a training dataset which can distinguish Cancer samples from Normal Control samples on the basis of y-score.
ii. Class Weight for the target variable were set in the AI model to overcome class imbalance in the training data.
iii. Optimization Algorithm were set in the AI model to handle complexity of data and making it faster.

This may generate a Cancer Detection AI (CDAI) Model that is capable of distinguishing cancer samples from normal controls.

The samples identified as cancer-positive by the CDAI algorithm are then subjected to analysis by second AI Model for tissue of origin identification (TOOAI Model) to distinguish between the individual cancers (e.g., lung cancer from the remaining 18 cancers). The TOOAI Model may either include a Support vector machine, Logistic one versus rest, or Stochastic gradient descent algorithms that serve as classifier models for training of the cancer samples.

Thus, a two-step modeling scheme may be applied on the test set, in an embodiment. That is, firstly, the CDAI model may be applied on the test set to differentiate cancer samples from normal samples. Then, the TOOAI model may be applied on the resulting predicted cancer samples to distinguish between the 18 individual cancers as well as the groups cancers termed as ‘others’. The 18 individual cancers are endometrial cancer, breast cancer, cervical cancer, ovarian cancer, lung cancer, leukemia, thyroid cancer, melanoma, colorectal cancer, kidney cancer, lymphoma, pancreatic cancer, liver & bile duct cancer, gastric cancer, head & neck cancer, esophageal cancer and prostate cancer. The TOOAI model may result in 18 scores for each sample, with each score defining probability of the respective sample belonging to one of the 18 classes.

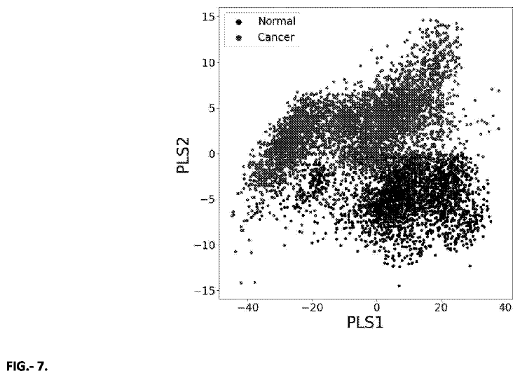

In a particular example, above process as implemented by the system 100 is performed: out of total 8971 samples 5057 samples were either endometrial cancer, breast cancer, cervical cancer, ovarian cancer, lung cancer, leukemia, thyroid cancer, melanoma, colorectal cancer, kidney cancer, lymphoma, pancreatic cancer, liver & bile duct cancer, gastric cancer, Larynx cancer, pharynx cancer, oral cancer, esophageal cancer, prostate cancer, bladder cancer, brain and CNS cancer, multiple myeloma, anus cancer, testicular cancer, vulva cancer, penile cancer, vagina cancer, gallbladder cancer, sarcoma cancer, germ cell tumor, squamous cell carcinoma and unknown primary or the remaining cancers grouped under the ‘others’ category. In addition, there were 3914 non-cancer controls. The data was randomly partitioned into training and test datasets in equal proportion. This resulted in 2479 cancer samples and 1957 non-cancer controls in the training set, and 2594 cancer samples and 1957 non-cancer controls in test set. The CDAI model was applied on the training set (See e.g., TABLE-3) and tested in the test set to obtain Accuracy, Sensitivity and Specificity values. While applying the CDAI model, a PLS DA regression function may be applied on the training dataset to find a function separating Cancer samples versus Normal Control samples FIG. 7.

Further, Class Weights for the target variables were set in the AI model to overcome class imbalance in the training data whereas an optimization algorithm was set in the AI model to handle complexity of data and making it faster. Thus, a CDAI may first be trained using the training dataset of samples. The resulting trained model/algorithm may find a score for each sample. Then, the trained CDAI model may be evaluated on a test set to determine the accuracy. The sensitivity, specificity and accuracy obtained in this example was 99.26%, 99.64%, and 99.8% respectively.

In yet another exemplary embodiment, the TOOAI model may be applied to the cancer-positive samples determined by the CDAI model. The TOOAI Model acted on the predicted cancers samples from the CDAI model and gave a multiclass score to each sample: one score for each cancer type as defined by its tissue of origin, denoting the probability of the sample belonging to the respective cancer type. Here, out of total 8971 cancer samples, 445 samples were endometrial cancer, 652 breast cancer, 458 cervical cancer, 488 ovarian cancer, 307 lung cancer, 157 leukemia, 169 thyroid cancer, 151 melanoma, 296 colorectal cancer, 136 kidney cancer, 97 lymphoma, 134 pancreatic cancer, 147 liver & bile duct cancer, 279 gastric cancer, 638 head & neck cancer, 143 esophageal cancer, and 122 prostate cancer and 254 samples in the category of ‘others’ TABLE-2. The data was randomly partitioned into training and test datasets in equal proportion. This resulted in case of 222 endometrial cancer, 326 breast cancer, 229 cervical cancer, 244 ovarian cancer, 153 lung cancer, 78 leukemia, 84 thyroid cancer, 75 melanoma cancer, 148 colorectal cancer, 68 kidney cancer, 48 lymphoma, 67 pancreatic cancer, 73 liver & bile duct cancer, 139 gastric cancer, 0 larynx cancer, 26 pharynx cancer, 283 oral cancer, 71 esophageal cancer, 61 prostate cancer, 16 bladder cancer, 21 brain & CNS cancer, 0 multiple myeloma, 0 anus cancer, 0 testicular cancer, 0 vulva cancer, 0 penile cancer, 0 vagina cancer, 17 gallbladder cancer, 22 sarcoma cancer, 0 germ cell tumor, 0 squamous cell carcinoma, 0 unknown primary, 1957 normal control and 8 samples in the category of ‘others’ in training set and in 223 endometrial cancer, 326 breast cancer, 229 cervical cancer, 244 ovarian cancer, 154 lung cancer, 79 leukemia, 85 thyroid cancer, 76 melanoma cancer, 148 colorectal cancer, 68 kidney cancer, 49 lymphoma, 67 pancreatic cancer, 74 liver & bile duct cancer, 140 gastric cancer, 20 larynx cancer, 26 pharynx cancer, 283 oral cancer, 72 esophageal cancer, 61 prostate cancer, 16 bladder cancer, 21 brain & CNS cancer, 18 multiple myeloma, 9 anus cancer, 4 testicular cancer, 5 vulva cancer, 18 penile cancer, 2 vagina cancer, 18 gallbladder cancer, 23 sarcoma cancer, 14 germ cell tumor, 8 squamous cell carcinoma, 6 unknown primary, 1957 normal control and 8 samples in the category of ‘others’ in test set TABLE-3 (shown in FIG. 3). Then, a Support vector machine, Logistic one versus rest, Stochastic gradient descent algorithms were used as classifier model on training samples to give the TOOAI model. Then, a two-step modeling scheme (CDAI Model followed by the TOOAI model) was applied on the test set. That is, the CDAI model first differentiated cancer from non-cancer samples in the test set. Then, the TOOAI Model was applied on the resulting predicted cancer samples. This resulted in 18 scores for each sample, with each score defining probability of the respective sample belonging to one of the 18 classes TABLE-2.

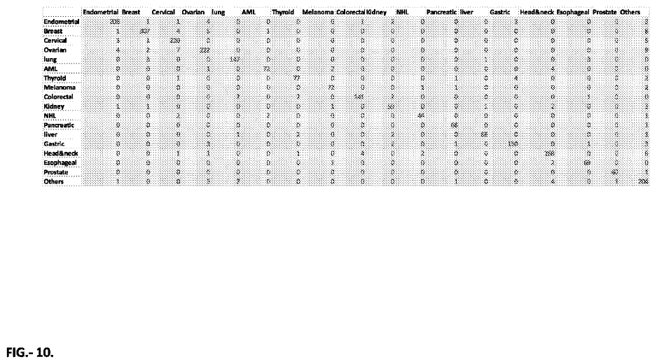

In some embodiments, the system 100 may be further implemented for determining the accuracy of the TOOAI model in differentiating specifically endometrial cancer from the remaining cancers within the 18-cancer group. The Endometrial cases were first differentiated from the normal control samples at 99.64% specificity, 100% sensitivity. The TOOAI model provides probability score for each cancer subclass for every given sample of endometrial cancer. This score can differentiate the cancer subclass of a sample. The top two scoring cancer subclasses were taken as the model result and were matched with the true labels. The final confusion matrix was built based on double class accuracy of the model. The endometrial cancer tissue identification Accuracy was calculated to be 92.6%. (See e.g., FIG. 10, TABLE-4).

In some embodiments, the system 100 may be further implemented for determining the accuracy of the TOOAI model in differentiating specifically Breast cancer from the remaining cancers within the 18-cancer group. The Breast cases were first differentiated from the normal control samples at 99.64% specificity, 100% sensitivity. The TOOAI model provides probability score for each cancer subclass for every given sample of Breast cancer. This score can differentiate the cancer subclass of a sample. The top two scoring cancer subclasses were taken as the model result and were matched with the true labels. The final confusion matrix was built based on double class accuracy of the model. The Breast cancer tissue identification Accuracy was calculated to be 93%. (See e.g., FIG. 10, TABLE-4).

In some embodiments, the system 100 may be further implemented for determining the accuracy of the TOOAI model in differentiating specifically Cervical cancer from the remaining cancers within the 18-cancer group. The Cervical cases were first differentiated from the normal control samples at 99.64% specificity, 99.6% sensitivity. The TOOAI model provides probability score for each cancer subclass for every given sample of Cervical cancer. This score can differentiate the cancer subclass of a sample. The top two scoring cancer subclasses were taken as the model result and were matched with the true labels. The final confusion matrix was built based on double class accuracy of the model. The Cervical cancer tissue identification Accuracy was calculated to be 96.6%. (See e.g., FIG. 10, TABLE-4). In some embodiments, the system 100 may be further implemented for determining the accuracy of the TOOAI model in differentiating specifically Ovarian cancer from the remaining cancers within the 18-cancer group. The Ovarian cases were first differentiated from the normal control samples at 99.64% specificity, 100% sensitivity. The TOOAI model provides probability score for each cancer subclass for every given sample of Ovarian cancer. This score can differentiate the cancer subclass of a sample. The top two scoring cancer subclasses were taken as the model result and were matched with the true labels. The final confusion matrix was built based on double class accuracy of the model. The Ovarian cancer tissue identification Accuracy was calculated to be 91%. (See e.g., FIG. 10, TABLE-4).

In some embodiments, the system 100 may be further implemented for determining the accuracy of the TOOAI model in differentiating specifically Lung cancer from the remaining cancers within the 18-cancer group. The Lung cases were first differentiated from the normal control samples at 99.64% specificity, 100% sensitivity. The TOOAI model provides probability score for each cancer subclass for every given sample of Lung cancer. This score can differentiate the cancer subclass of a sample. The top two scoring cancer subclasses were taken as the model result and were matched with the true labels. The final confusion matrix was built based on double class accuracy of the model. The Lung cancer tissue identification Accuracy was calculated to be 93%. (See e.g., FIG. 10, TABLE-4).

In some embodiments, the system 100 may be further implemented for determining the accuracy of the TOOAI model in differentiating specifically leukemia from the remaining cancers within the 18-cancer group. The leukemia cases were first differentiated from the normal control samples at 99.64% specificity, 100% sensitivity. The TOOAI model provides probability score for each cancer subclass for every given sample of leukemia. This score can differentiate the cancer subclass of a sample. The top two scoring cancer subclasses were taken as the model result and were matched with the true labels. The final confusion matrix was built based on double class accuracy of the model. The leukemia tissue identification Accuracy was calculated to be 83.3%. (See e.g., FIG. 10, TABLE-4).

In some embodiments, the system 100 may be further implemented for determining the accuracy of the TOOAI model in differentiating specifically Thyroid cancer from the remaining cancers within the 18-cancer group. The Thyroid cases were first differentiated from the normal control samples at 99.64% specificity, 100% sensitivity. The TOOAI model provides probability score for each cancer subclass for every given sample of Thyroid cancer. This score can differentiate the cancer subclass of a sample. The top two scoring cancer subclasses were taken as the model result and were matched with the true labels. The final confusion matrix was built based on double class accuracy of the model. The Thyroid cancer tissue identification Accuracy was calculated to be 87.5%. (See e.g., FIG. 10, TABLE-4).

In some embodiments, the system 100 may be further implemented for determining the accuracy of the TOOAI model in differentiating specifically Melanoma from the from the remaining cancers within the 18-cancer group. The Melanoma cases were first differentiated from the normal control samples at 99.64% specificity, 100% sensitivity. The TOOAI model provides probability score for each cancer subclass for every given sample of Melanoma. This score can differentiate the cancer subclass of a sample. The top two scoring cancer subclasses were taken as the model result and were matched with the true labels. The final confusion matrix was built based on double class accuracy of the model. The Melanoma tissue identification Accuracy was calculated to be 92.8%. (See e.g., FIG. 10, TABLE-4).

In some embodiments, the system 100 may be further implemented for determining the accuracy of the TOOAI model in differentiating specifically Colorectal cancer from the remaining cancers within the 18-cancer group. The Colorectal cases were first differentiated from the normal control samples at 99.64% specificity, 100% sensitivity. The TOOAI model provides probability score for each cancer subclass for every given sample of Colorectal cancer. This score can differentiate the cancer subclass of a sample. The top two scoring cancer subclasses were taken as the model result and were matched with the true labels. The final confusion matrix was built based on double class accuracy of the model. The Colorectal tissue identification cancer Accuracy was calculated to be 92.5%. (See e.g., FIG. 10, TABLE-4).

In some embodiments, the system 100 may be further implemented for determining the accuracy of the TOOAI model in differentiating specifically Kidney cancer from the remaining cancers within the 18-cancer group. The Kidney cases were first differentiated from the normal control samples at 99.64% specificity, 100% sensitivity. The TOOAI model provides probability score for each cancer subclass for every given sample of Kidney cancer. This score can differentiate the cancer subclass of a sample. The top two scoring cancer subclasses were taken as the model result and were matched with the true labels. The final confusion matrix was built based on double class accuracy of the model. The Kidney cancer tissue identification Accuracy was calculated to be 86%. (See e.g., FIG. 10, TABLE-4).

In some embodiments, the system 100 may be further implemented for determining the accuracy of the TOOAI model in differentiating specifically lymphoma from the remaining cancers within the 18-cancer group. The Lymphoma cases were first differentiated from the normal control samples at 99.64% specificity, 100% sensitivity. The TOOAI model provides probability score for each cancer subclass for every given sample of lymphoma. This score can differentiate the cancer subclass of a sample. The top two scoring cancer subclasses were taken as the model result and were matched with the true labels. The final confusion matrix was built based on double class accuracy of the model. The lymphoma tissue identification Accuracy was calculated to be 89%. (See e.g., FIG. 10, TABLE-4).

In some embodiments, the system 100 may be further implemented for determining the accuracy of the TOOAI model in differentiating specifically Pancreatic cancer from the remaining cancers within the 18-cancer group. The Pancreatic cases were first differentiated from the normal control samples at 99.64% specificity, 100% sensitivity. The TOOAI model provides probability score for each cancer subclass for every given sample of Pancreatic cancer. This score can differentiate the cancer subclass of a sample. The top two scoring cancer subclasses were taken as the model result and were matched with the true labels. The final confusion matrix was built based on double class accuracy of the model. The Pancreatic cancer tissue identification Accuracy was calculated to be 100%. (See e.g., FIG. 10, TABLE-4).

In some embodiments, the system 100 may be further implemented for determining the accuracy of the TOOAI model in differentiating specifically liver cancer from the remaining cancers within the 18-cancer group. The liver cases were first differentiated from the normal control samples at 99.64% specificity, 100% sensitivity. The TOOAI model provides probability score for each cancer subclass for every given sample of liver cancer. This score can differentiate the cancer subclass of a sample. The top two scoring cancer subclasses were taken as the model result and were matched with the true labels. The final confusion matrix was built based on double class accuracy of the model. The liver cancer Accuracy was calculated to be 80%. (See e.g., FIG. 10, TABLE-4).

In some embodiments, the system 100 may be further implemented for determining the accuracy of the TOOAI model in differentiating specifically Gastric cancer from the remaining cancers within the 18-cancer group. The Gastric cases were first differentiated from the normal control samples at 99.64% specificity, 100% sensitivity. The TOOAI model provides probability score for each cancer subclass for every given sample of Gastric cancer. This score can differentiate the cancer subclass of a sample. The top two scoring cancer subclasses were taken as the model result and were matched with the true labels. The final confusion matrix was built based on double class accuracy of the model. The Gastric cancer tissue identification Accuracy was calculated to be 82%. (See e.g., FIG. 10, TABLE-4).

In some embodiments, the system 100 may be further implemented for determining the accuracy of the TOOAI model in differentiating specifically head & neck cancer from the remaining cancers within the 18-cancer group. The head & neck cases were first differentiated from the normal control samples at 99.64% specificity, 100% sensitivity. The TOOAI model provides probability score for each cancer subclass for every given sample of head & neck cancer. This score can differentiate the cancer subclass of a sample. The top two scoring cancer subclasses were taken as the model result and were matched with the true labels. The final confusion matrix was built based on double class accuracy of the model. The head & neck cancer tissue identification Accuracy was calculated to be 94%. (See e.g., FIG. 10, TABLE-4).

In some embodiments, the system 100 may be further implemented for determining the accuracy of the TOOAI model in differentiating specifically esophageal cancer from the remaining cancers within the 18-cancer group. The head & neck cases were first differentiated from the normal control samples at 99.64% specificity, 100% sensitivity. The TOOAI model provides probability score for each cancer subclass for every given sample of head & neck cancer. This score can differentiate the cancer subclass of a sample. The top two scoring cancer subclasses were taken as the model result and were matched with the true labels. The final confusion matrix was built based on double class accuracy of the model. The head & neck cancer tissue identification Accuracy was calculated to be 87%. (See e.g., FIG. 10, TABLE-4).

In some embodiments, the system 100 may be further implemented for determining the accuracy of the TOOAI model in differentiating specifically prostate cancer from the remaining cancers within the 18-cancer group. The head & neck cases were first differentiated from the normal control samples at 99.64% specificity, 100% sensitivity. The TOOAI model provides probability score for each cancer subclass for every given sample of head & neck cancer. This score can differentiate the cancer subclass of a sample. The top two scoring cancer subclasses were taken as the model result and were matched with the true labels. The final confusion matrix was built based on double class accuracy of the model. The head & neck cancer tissue identification Accuracy was calculated to be 87.5%. (See e.g., FIG. 10, TABLE-4).

In some embodiments, the system 100 may be further implemented for determining the accuracy of the TOOAI model in differentiating specifically “others” cancer from the remaining cancers within the 18-cancer group. The head & neck cases were first differentiated from the normal control samples at 99.64% specificity, 99.09% sensitivity. The TOOAI model provides probability score for each cancer subclass for every given sample of head & neck cancer. This score can differentiate the cancer subclass of a sample. The top two scoring cancer subclasses were taken as the model result and were matched with the true labels. The final confusion matrix was built based on double class accuracy of the model. The head & neck cancer tissue identification Accuracy was calculated to be 94%. (See e.g., FIG. 10, TABLE-4).

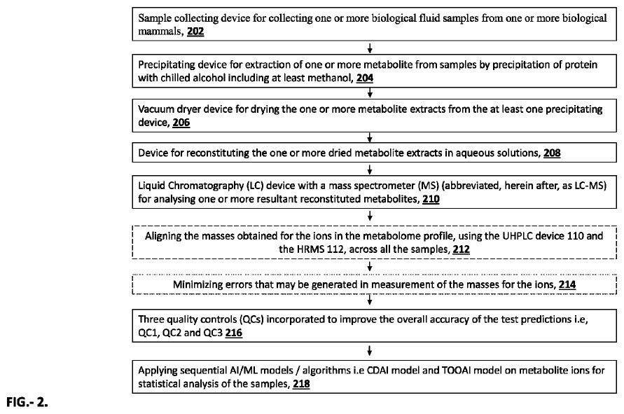

Referring to FIG. 2 that illustrates a flow chart for implementing metabolomics process for differentiating the cancer samples (for example, endometrial cancer, breast cancer, cervical cancer, ovarian cancer, lung cancer, leukemia, thyroid cancer, melanoma, colorectal cancer, kidney cancer, lymphoma, pancreatic cancer, liver & bile duct cancer, gastric cancer, Larynx cancer, pharynx cancer, oral cancer, esophageal cancer, prostate cancer, bladder cancer, brain and CNS cancer, multiple myeloma, anus cancer, testicular cancer, vulva cancer, penile cancer, vagina cancer, gallbladder cancer, sarcoma cancer, germ cell tumor, squamous cell carcinoma and unknown primary, and the additional cancers grouped under the category of ‘others’) from the normal controls and further to identify each specific cancer type of each sample from that belonging to the other cancer types, in accordance with an embodiment of the present invention. The FIG. 2 should be read and understood in conjunction with the FIGS. 1 and 3-11, and also may include at least one or more embodiments of the FIGS. 1 and 3-11, without deviating from the meaning and scope of the present invention.

Further, the method 200 may include at least one or more steps 202-218, individually or in combination.

Also, the method 200 is explained by taking an example of multiple cancers including endometrial cancer, breast cancer, cervical cancer, ovarian cancer, lung cancer, leukemia, thyroid cancer, melanoma, colorectal cancer, kidney cancer, lymphoma, pancreatic cancer, liver & bile duct cancer, gastric cancer, head & neck cancer, esophageal cancer, prostate cancer, and the additional cancers grouped in the ‘others’ category, and should not be considered to limit the meaning and scope of the present invention.

The FIG. 2 shows a metabolomics process 200 that may include a step 202 for collecting and storing number of samples from male and female volunteers who are either free of any cancer (normal control) (n=3914), or have endometrial cancer (n=445), breast cancer (n=652), cervical cancer (n=458), ovarian cancer (n=488), lung cancer (n=307), leukemia (n=157), thyroid cancer (n=169), melanoma (n=151), colorectal cancer (n=296), kidney cancer (n==136), lymphoma (n=97), pancreatic cancer (n=134), liver & bile duct cancer (n=147), gastric cancer (n=279), larynx cancer (n=20), pharynx cancer (n=52), oral cancer (n=566), esophageal cancer (n=143), prostate cancer (n=122), bladder (n=32), brain and CNS cancer (n=42), multiple myeloma (n=18), anus cancer (n=9), testicular cancer (n=4), vulva cancer (n=5), penile cancer (n=18), vagina cancer (n=2), gallbladder cancer (n=35), sarcoma cancer (n=45), germ cell tumor (n=14), squamous cell carcinoma (n=8), unknown primary (n=6) and other cancers (n=16) TABLE-1. The samples are collected and stored in the sample collecting device 102.

Further, the method includes a step 204 extracting a metabolite extraction which may be achieved by precipitating serum proteins with chilled methanol. In an embodiment, the precipitation device 104 may be a test tube. The supernatant may be collected as the metabolite extract. Thereafter, at a step 206, the metabolite extract may be dried before use. For the process of drying, in an embodiment, the phase separation device 106 may be used that may dry the metabolite extract using speed vacuum. At a step of 208, in an embodiment, the dried extract may be reconstituted in an aqueous solution in a mobile phase using a device 108. Thereafter, at a step of 210, analysis of the resultant samples, derived from the reconstitution phase, may be performed by the LCMS 110. At step 210, the reconstituted samples may be first resolved by Liquid Chromatography (abbreviated as LC) device 110, and then, the ion spectra may be subsequently obtained through high-resolution mass spectrometer (abbreviated as MS). Using the LCMS device 110, ions in the metabolite extraction may be measured, the masses for the ions may be measured based on their mass-to-charge ratio or m/z.

Thereafter, the features of the ion spectra accumulated in metabolic profile may be extracted using the computing device 112 that may execute, using one or more processors, compound discoverer software. Furthermore, in an embodiment, the method 200 may include a step of 212 aligning the masses obtained for the ions in the metabolome profile, using the LCMS, across all the samples. This may be done to enable comparison of the peak intensity of each ion across all the samples.

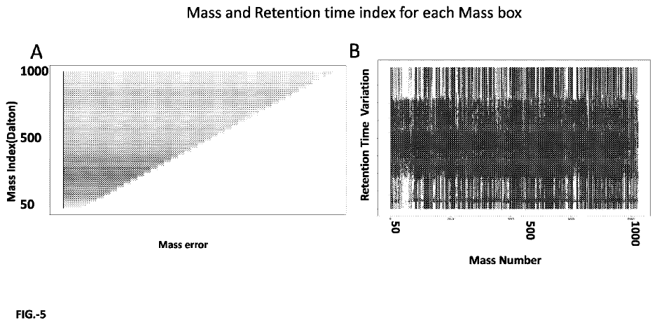

Further, in an embodiment, additional optional step 214 included to minimize the errors that may be generated in measurement of the masses for the ions. To normalize for unavoidable, but minor, variations in mass (m/z), a sophisticated approach of using parts per million (ppm) error-based approach may be used, according to an embodiment. In another embodiment, briefly, a modified virtual lock mass-based approach may also be used. This is based on the principle that mass errors are known to increase with mass. This modified virtual lock mass-based approach may be used and adapted according to the datasets in examples of the invention. This may be done by combining the traditional virtual lock mass approach with metabolite identification from the Human Metabolome Database (HMDB). Specifically, the virtual lock mass boxes may be defined using the masses of metabolites identified by HMDB database search across multiple samples FIG. 5.

Thereafter a step of 216 applied on the aligned and normalized ion spectra to further improve the overall accuracy of the algorithm used for prediction. The step 216 comprises of three quality checks (QCs) that are explained as follows.

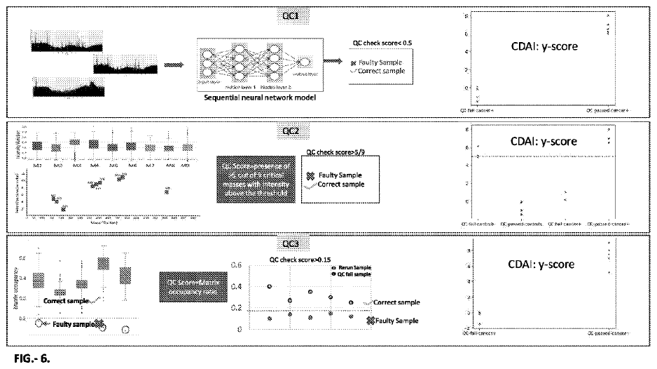

Step QC1 of System 114 involves Chromatogram profile matching. A faulty chromatogram may be a result of faulty sample extraction, or due to an error in the mass spectrometry setting. These errors impact on the quality of data, which then compromise the overall prediction accuracies of the algorithms. A sequential neural network model was built to detect these faults based on variations in the chromatogram profiles. The chromatogram obtained from the mass spectrometer for each sample were first converted into jpeg format. The image was then scaled to appropriate width and length for the model training. The Image was binarized in order to segregate the chromatogram, which facilitates more efficient analysis. Keras Sequential neural network model was then used to train-test validate the model. We employed three Keras Conv2D 2D Convolution Layers, which create a convolution kernel that is wound with layered inputs that help produce a tensor of outputs. An 80-20 train-test split was taken. Adam optimizer was subsequently used to update network weights iteratively based on the training data. The threshold used for the correct detection was 0.5 and samples which showed a Quality Control (QC) score of <0.5 were designated as having passed QC1. Samples with >0.5 QC score were rejected as having failed at the QC1 step. We obtained 100% detection accuracy for faulty chromatograms as shown in FIG. 6 A.

Step QC2 of System 114 monitors for the presence of critical m/z ions. The second step of System 114, which is called QC2, monitored for the presence of at least 6 out of 9 critical ions with m/z values ranging from 100 to 800. The distribution of intensity and RT of these 9 ions is shown in the FIG. 6 B. Presence of 6 or more of the 9 critical ions in the chromatogram of a sample is taken as the threshold criteria for passing the QC2 Step. Samples with <6 out of the 9 critical masses are rejected as having failed the QC2 step. The QC2 step is important for the accurate identification of cancer samples because those samples that do not pass this step have a higher likelihood of misclassification by the CDAI algorithm (FIG. 6 B).

Step QC3 of System 114 monitors matrix occupancy: Another layer of quality check that was introduced along with previous two quality checks involved an assessment of matrix occupancy. This layer is Step QC3 and it relies on the percentage of features that matches with the matrix size. The threshold was optimized with multiple runs of poor-quality samples, or with improper mass spectrometry run conditions. Based on these studies the threshold for minimum matrix occupancy was set at 15%. This threshold was confirmed through multiple validation exercises, which was then found to improve the robustness and accuracy of prediction CDAI algorithm. With the trained model for QC3, faulty samples could be captured with 100% accuracy as shown in FIG. 6 C.

Furthermore, in an embodiment, the method 200 may include a step of 218 that may use one or more AI/ML algorithms for AI based pattern recognition for final identifying, differentiating and presenting the cancer samples from the normal control samples and further to identify, differentiate and present individual cancer samples within the identified cancer samples.

The method 200 may furthermore include a step 218 of applying AI/ML models/algorithms on the obtained, measured (also, e.g., aligned, corrected) and featured metabolite ions, which are measured and aligned as explained above. The step 218 may include applying AI/ML models for statistical analysis of the samples. The computing device 116 that may be able to execute one or more AI/ML algorithms for applying the AI/ML models for statistical analysis of the samples.

The step 218 of applying AI/ML models/algorithms may include creating and applying at least two AI models, namely first the CDAI Model and followed by the TOOAI Model. By executing the one or more AI/ML algorithms, using one or more processors, at the computing device 116, one or more first AI/ML models may be generated to distinguish the cancer samples (endometrial cancer, breast cancer, cervical cancer, ovarian cancer, lung cancer, leukemia, thyroid cancer, melanoma, colorectal cancer, kidney cancer, lymphoma, pancreatic cancer, liver & bile duct cancer, gastric cancer, Larynx cancer, pharynx cancer, oral cancer, esophageal cancer, prostate cancer, bladder cancer, brain and CNS cancer, multiple myeloma, anus cancer, testicular cancer, vulva cancer, penile cancer, vagina cancer, gallbladder cancer, sarcoma cancer, germ cell tumor, squamous cell carcinoma and unknown primary and the additional cancers grouped as ‘others’) from the normal controls. Further, in another embodiment, executing the one or more AI/ML algorithms, using one or more processors, at the computing device 116, may be followed by a second AI/ML model to further distinguish and identify the cancer type as defined by its tissue of origin (e.g., colorectal cancer from the remaining cancer types) TABLE-2.

The step 218 may be optionally included in the method 200. Further, the flow of the steps 202-218 may be altered, and may not be restricted to as shown in the method 200. While generating the AI/Models at step 218, that may follow one or more of the following steps:
i. While developing the AI model, a functional mapping is established in between dependent/target variable and independent variable learning in a training dataset which can distinguish Cancer samples from Normal Control samples on the basis of y-score.
ii. Class Weight for the target variable were set in the AI model to overcome class imbalance in the training data.
iii. Optimization Algorithm were set in the AI model to handle complexity of data and making it faster.

This may generate CDAI Model, at the step 218, that may separate normal control samples from cancer Samples. Another AI Model, termed as the TOOAI Model, may also be generated, at step 218 and applied to cancer-positive samples identified by the CDAI Model to distinguish the individual cancer type (e.g., colorectal cancer) from the remaining cancer types (e.g., endometrial cancer, breast cancer, cervical cancer, ovarian cancer, lung cancer, leukemia, thyroid cancer, melanoma, kidney cancer, lymphoma, pancreatic cancer, liver & bile duct cancer, gastric cancer, head & neck cancer, esophageal cancer, prostate cancer, and the group of ‘other’ cancers) TABLE-2 from the normal controls. In an embodiment, the TOOAI Model may be generated in a similar way as the CDAI, and may further include a Support vector machine, Logistic one versus rest, Stochastic gradient descent algorithms classifier that act as a classification model that may be made using the training samples to give the second TOOAI FIG. 8.

Thus, a two-step modeling scheme may be applied on the test set, in an embodiment. That is, firstly, the CDAI Model to differentiate cancer samples from normal samples may be applied on the test set. Then, the TOOAI may be applied on the resulting predicted cancer samples. Now, if 18 cancer types are taken, for example endometrial cancer, breast cancer, cervical cancer, ovarian cancer, lung cancer, leukemia, thyroid cancer, melanoma, kidney cancer, lymphoma, pancreatic cancer, liver & bile duct cancer, colorectal cancer, gastric cancer, head & neck cancer, esophageal cancer, prostate cancer and the additional cancers grouped as ‘others’, then this two-step modeling scheme may result in 18 scores for each sample, with each score defining probability of the respective sample belonging to one of the 18 classes.

The present invention is illustrated by examples. The examples are meant only for illustrative purposes and should not be construed as limiting. The examples below are described in detail, while implementing the system and method described in FIGS. 1-2, respectively.

### Examples

Using liquid chromatography-mass spectrometry for untargeted metabolomics of serum described the approach for early-stage distinction of multiple cancer both in male and female from the control cases. The specimen collected for these cancer subject i.e., male and female volunteers who are either free of any cancer (normal control) (n=3914), or have endometrial cancer (n=445), breast cancer (n=652), cervical cancer (n=458), ovarian cancer (n=488), lung cancer (n=307), leukemia (n=157), thyroid cancer (n=169), melanoma (n=151), colorectal cancer (n=296), kidney cancer (n=136), lymphoma (n=97), pancreatic cancer (n=134), liver & bile duct cancer (n=147), gastric cancer (n=279), larynx cancer (n=20), pharynx cancer (n=52), oral cancer (n=566), esophageal cancer (n=143), prostate cancer (n=122), bladder (n=32), brain and CNS cancer (n=42), multiple myeloma (n=18), anus cancer (n=9), testicular cancer (n=4), vulva cancer (n=5), penile cancer (n=18), vagina cancer (n=2), gallbladder cancer (n=35), sarcoma cancer (n=45), germ cell tumor (n=14), squamous cell carcinoma (n=8), unknown primary (n=6) and other cancers (n=16) TABLE-1. Specimen collected for normal control cases was 3914 in number.

TABLE 1
Details of the specimen collected

| | | | | | | | | BMI | | | | | | | | |
|---|---|---|---|---|---|---|---|---|---|---|---|---|---|---|---|---|
| | | | | | | | | (kg/m2) | | Ethnicity | | | | | | |
| | Age Distribution (Years) | | | | | | | 10 to | | | Non- | Cancer Stages | | | | |
| | 20-30 | 31-40 | 41-50 | 51-60 | 61-70 | 71-80 | 81-90 | 30 | >30 | white | white | 0 | I | II | III | IV |
| Normal control | 702 | 735 | 832 | 823 | 596 | 189 | 37 | 3310 | 481 | 203 | 3711 | 0 | 0 | 0 | 0 | 0 |
| (n = 3914) | | | | | | | | | | | | | | | | |
| Endometrial | 2 | 12 | 89 | 209 | 107 | 22 | 4 | 250 | 193 | 302 | 143 | 0 | 350 | 64 | 23 | 8 |
| cancer | | | | | | | | | | | | | | | | |
| (n = 445) | | | | | | | | | | | | | | | | |
| Breast cancer | 20 | 109 | 183 | 212 | 94 | 28 | 6 | 582 | 61 | 246 | 406 | 36 | 335 | 179 | 80 | 22 |
| (n = 652) | | | | | | | | | | | | | | | | |
| Cervical | 21 | 103 | 126 | 114 | 63 | 27 | 4 | 281 | 37 | 233 | 225 | 70 | 236 | 92 | 43 | 17 |
| cancer | | | | | | | | | | | | | | | | |
| (n = 458) | | | | | | | | | | | | | | | | |
| Ovarian | 18 | 82 | 144 | 139 | 87 | 13 | 5 | 296 | 66 | 256 | 232 | 19 | 292 | 96 | 59 | 22 |
| cancer | | | | | | | | | | | | | | | | |
| (n = 488) | | | | | | | | | | | | | | | | |
| Lung cancer | 12 | 22 | 54 | 119 | 76 | 21 | 3 | 249 | 19 | 80 | 227 | 7 | 112 | 55 | 80 | 53 |
| (n = 307) | | | | | | | | | | | | | | | | |
| Leukemia | 23 | 12 | 30 | 44 | 30 | 15 | 3 | 92 | 14 | 71 | 86 | 0 | 10 | 15 | 29 | 32 |
| (n = 157) | | | | | | | | | | | | | | | | |
| Thyroid cancer | 24 | 33 | 44 | 39 | 21 | 6 | 2 | 118 | 16 | 70 | 99 | 0 | 83 | 49 | 32 | 5 |
| (n = 169) | | | | | | | | | | | | | | | | |
| Melanoma | 4 | 16 | 25 | 48 | 38 | 14 | 6 | 102 | 18 | 86 | 65 | 2 | 82 | 42 | 18 | 7 |
| cancer (n = 151) | | | | | | | | | | | | | | | | |
| Colorectal | 14 | 34 | 60 | 90 | 61 | 30 | 7 | 280 | 16 | 87 | 209 | 0 | 82 | 100 | 95 | 19 |
| cancer (n = 296) | | | | | | | | | | | | | | | | |
| Kidney cancer | 2 | 10 | 19 | 39 | 56 | 9 | 1 | 101 | 34 | 80 | 56 | 0 | 77 | 30 | 21 | 8 |
| (n = 136) | | | | | | | | | | | | | | | | |
| Lymphoma | 9 | 12 | 15 | 24 | 19 | 16 | 2 | 77 | 19 | 50 | 47 | 0 | 13 | 15 | 9 | 10 |
| (n = 97) | | | | | | | | | | | | | | | | |
| Pancreatic | 1 | 6 | 20 | 31 | 60 | 15 | 1 | 120 | 14 | 75 | 59 | 0 | 32 | 77 | 17 | 8 |
| cancer (n = 134) | | | | | | | | | | | | | | | | |
| Liver & bile | 3 | 18 | 38 | 50 | 28 | 10 | 0 | 124 | 6 | 34 | 113 | 0 | 62 | 41 | 32 | 12 |
| cancer | | | | | | | | | | | | | | | | |
| (n = 147) | | | | | | | | | | | | | | | | |
| Gastric cancer | 5 | 26 | 61 | 78 | 67 | 36 | 6 | 257 | 18 | 85 | 194 | 0 | 103 | 110 | 57 | 9 |
| (n = 279) | | | | | | | | | | | | | | | | |
| Larynx cancer | 1 | 1 | 4 | 4 | 6 | 2 | 2 | 19 | 1 | 0 | 20 | 0 | 7 | 8 | 2 | 3 |
| (n = 20) | | | | | | | | | | | | | | | | |
| Pharynx | 5 | 4 | 9 | 11 | 14 | 9 | 0 | 48 | 4 | 10 | 42 | 0 | 16 | 22 | 9 | 5 |
| cancer (n = 52) | | | | | | | | | | | | | | | | |
| Oral cancer | 22 | 96 | 140 | 167 | 99 | 35 | 7 | 520 | 20 | 78 | 488 | 14 | 105 | 258 | 136 | 53 |
| (n = 566) | | | | | | | | | | | | | | | | |
| Oesophageal | 3 | 13 | 33 | 41 | 39 | 14 | 0 | 143 | 0 | 0 | 143 | 0 | 26 | 61 | 42 | 14 |
| cancer (n = 143) | | | | | | | | | | | | | | | | |
| Prostate | 0 | 3 | 14 | 30 | 50 | 24 | 1 | 119 | 3 | 0 | 122 | 0 | 17 | 37 | 55 | 13 |
| cancer (n = 122) | | | | | | | | | | | | | | | | |
| Bladder cancer | 0 | 5 | 5 | 3 | 14 | 5 | 0 | 32 | 0 | 0 | 32 | 0 | 4 | 12 | 14 | 2 |
| (n = 32) | | | | | | | | | | | | | | | | |
| Brain & | 12 | 12 | 10 | 7 | 1 | 0 | 0 | 42 | 0 | 0 | 42 | 0 | 11 | 13 | 14 | 4 |
| central | | | | | | | | | | | | | | | | |
| nervous system | | | | | | | | | | | | | | | | |
| cancer (n = 42) | | | | | | | | | | | | | | | | |
| Multiple | 0 | 0 | 3 | 6 | 5 | 4 | 0 | 18 | 0 | 0 | 18 | 0 | 0 | 7 | 7 | 4 |
| Myeloma | | | | | | | | | | | | | | | | |
| cancer (n = 18) | | | | | | | | | | | | | | | | |
| Anus cancer | 0 | 3 | 3 | 2 | 10 | 10 | 1 | 9 | 0 | 0 | 9 | 0 | 2 | 3 | 3 | 1 |
| (n = 9) | | | | | | | | | | | | | | | | |
| Testicular | 2 | 0 | 1 | 1 | 0 | 0 | 0 | 4 | 0 | 0 | 4 | 0 | 0 | 2 | 2 | 0 |
| cancer (n = 4) | | | | | | | | | | | | | | | | |
| Vulva cancer | 1 | 0 | 2 | 1 | 1 | 0 | 0 | 5 | 0 | 0 | 5 | 0 | 0 | 1 | 2 | 2 |
| (n = 5) | | | | | | | | | | | | | | | | |
| Penile cancer | 0 | 2 | 5 | 3 | 5 | 3 | 0 | 18 | 0 | 0 | 18 | 0 | 4 | 7 | 7 | 0 |
| (n = 18) | | | | | | | | | | | | | | | | |
| Vagina cancer | 0 | 0 | 0 | 1 | 1 | 0 | 0 | 2 | 0 | 0 | 2 | 0 | 0 | 0 | 2 | 0 |
| (n = 2) | | | | | | | | | | | | | | | | |
| Gall Bladder | 1 | 3 | 10 | 10 | 10 | 1 | 0 | 35 | 0 | 0 | 35 | 0 | 3 | 18 | 13 | 1 |
| cancer (n = 35) | | | | | | | | | | | | | | | | |
| Sarcoma | 12 | 11 | 9 | 9 | 3 | 1 | 0 | 44 | 1 | 0 | 45 | 0 | 6 | 10 | 23 | 6 |
| (n = 45) | | | | | | | | | | | | | | | | |
| Germ Cell | 1 | 6 | 3 | 4 | 0 | 0 | 0 | 14 | 0 | 0 | 14 | 0 | 0 | 10 | 3 | 1 |
| Tumor (n = 14) | | | | | | | | | | | | | | | | |
| Squamous cell | 0 | 1 | 3 | 4 | 0 | 0 | 0 | 8 | 0 | 0 | 8 | 0 | 3 | 1 | 3 | 1 |
| carcinoma | | | | | | | | | | | | | | | | |
| (n = 8) | | | | | | | | | | | | | | | | |
| unknown | 0 | 0 | 2 | 1 | 2 | 1 | 0 | 6 | 0 | 0 | 6 | | | | | |
| Primary (n = 6) | | | | | | | | | | | | | | | | |
| Others (n = 16) | 3 | 2 | 4 | 2 | 3 | 1 | 1 | 16 | 0 | 0 | 16 | 0 | 0 | 4 | 4 | 1 |

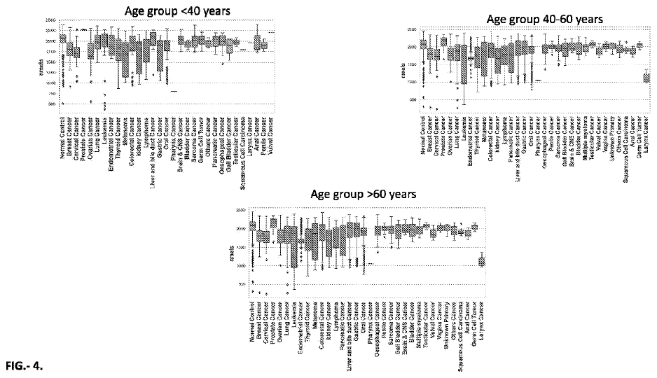

The untargeted metabolomics approach (See e.g., FIG. 2) generated a large metabolites list in female cases, which were further divided into subset of normal control, endometrial cancer, breast cancer, cervical cancer, ovarian cancer, lung cancer, leukemia, thyroid cancer, melanoma, colorectal cancer, kidney cancer, lymphoma, pancreatic cancer, liver & bile duct cancer, gastric cancer, Larynx cancer, pharynx cancer, oral cancer, esophageal cancer, prostate cancer, bladder cancer, brain and CNS cancer, multiple myeloma, anus cancer, testicular cancer, vulva cancer, penile cancer, vagina cancer, gallbladder cancer, sarcoma cancer, germ cell tumor, squamous cell carcinoma and unknown primary, with 1704, 1821, 1766, 1762, 1846, 1481, 1725, 1605, 1780, 1578, 1613, 1655, 1826, 1770, 1164, 1408, 1845, 1940, 2095, 1968, 2016, 1973, 1933, 2025, 1954, 1948, 2014, 1877, 1959, 2027, 1911, and 1903, respectively, while “other” cancer has total of 1915 metabolites union across all the sample of a subclass respectively (See e.g., FIG. 4). The total number of unique metabolites identified in the present study was 2709 in number. The plant and drug metabolites were removed from this database.

Next, the data was passed through our data processing pipeline (See e.g., FIG. 8). Briefly, here firstly samples were aligned using a combination of VLM approach along with identified metabolites to make a matrix of 8971 samples and 2709 metabolites along with the corresponding intensity information. This intensity values were transformed into log 10 scale. Then, in an embodiment, metabolites ion filtering was performed to eliminate metabolites having weightage below the threshold value obtained from the PLS-DA regression mapping of cancer vs control samples. Then, in an embodiment, data normalization and missing value imputation were performed on the data. This resulted in a matrix of total of 2709 metabolites across 8971 samples. Out of 5057 samples, 445 samples were endometrial cancer, 652 breast cancer, 458 cervical cancer, 488 ovarian cancer, 307 lung cancer, 157 leukemia, 169 thyroid cancer, 151 melanoma, 296 colorectal cancer, 136 kidney cancer, 97 lymphoma, 134 pancreatic cancer, 147 liver & bile duct cancer, 279 gastric cancer, 638 head & neck cancer, 143 esophageal cancer, 122 prostate cancer and 3914 were normal control samples TABLE-2.

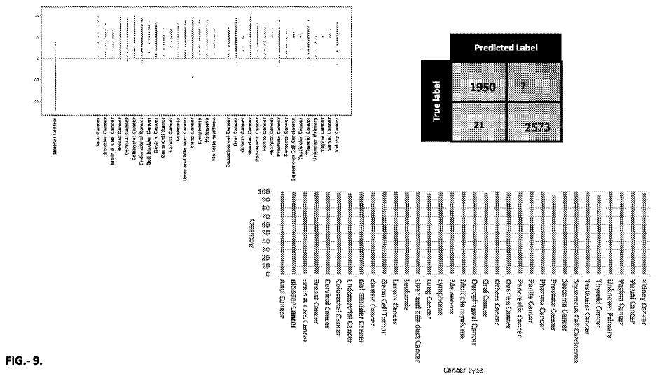

To find whether there was any difference in these samples based on the metabolite profiles, the matrix generated above was used. A PLS DA plot was made using the matrix as shown in FIG. 7. The FIG. 7 clearly shows that each cancer can be distinguished from healthy samples based on their metabolic data in case of both male and female. To quantify how well these can be distinguished, an AI analysis (See e.g., FIGS. 1-2, FIG. 9, FIG. 10, TABLE-4) was done on the data as described below to find common patterns in metabolite variations within cancer samples which is different from control samples. Furthermore, a classification model built on the detected metabolite ions with random distribution of samples into testing and training sets (See e.g., FIGS. 1-2, FIG. 9, FIG. 10, TABLE-4). The first such model (the CDAI Model) was built to distinguish between cancer and normal control sample. For this exercise, 5057 cancer samples and 3914 normal control cases were taken into consideration. Further, these study samples (n=8971) were randomly divided (50%) into training and testing sets (See e.g., TABLE-3). A multivariate classifier was derived into the training set and evaluated in the testing sets and a confusion matrix with predicted and true label was generated. This leads to ultimately, distinguish cancer samples from the controls with 100%, 99.64% and 100% of sensitivity, specificity and accuracy respectively (See e.g., FIG. 9).

Further, a multiclass classifier was also built to distinguish cancers from each other. Here, a model (the TOOAI Model) was built with total of 445 samples were endometrial cancer, 652 breast cancer, 458 cervical cancer, 488 ovarian cancer, 307 lung cancer, 157 leukemia, 169 thyroid cancer, 151 melanoma, 296 colorectal cancer, 136 kidney cancer, 97 lymphoma, 134 pancreatic cancer, 147 liver & bile duct cancer, 279 gastric cancer, 638 head & neck cancer, 143 esophageal cancer, 122 prostate cancer and 3914 were normal control samples TABLE-2. These study samples were randomly divided (50%) into the training and testing sets. This resulted in case of 222 endometrial cancer, 326 breast cancer, 229 cervical cancer, 244 ovarian cancer, 153 lung cancer, 78 leukemia, 84 thyroid cancer, 75 melanoma cancer, 148 colorectal cancer, 68 kidney cancer, 48 lymphoma, 67 pancreatic cancer, 73 liver & bile duct cancer, 139 gastric cancer, 0 larynx cancer, 26 pharynx cancer, 283 oral cancer, 71 esophageal cancer, 61 prostate cancer, 16 bladder cancer, 21 brain & CNS cancer, 0 multiple myeloma, 0 anus cancer, 0 testicular cancer, 0 vulva cancer, 0 penile cancer, 0 vagina cancer, 17 gallbladder cancer, 22 sarcoma cancer, 0 germ cell tumor, 0 squamous cell carcinoma, 0 unknown primary, 1957 normal control and 8 samples in the category of ‘others’ in training set and in 223 endometrial cancer, 326 breast cancer, 229 cervical cancer, 244 ovarian cancer, 154 lung cancer, 79 leukemia, 85 thyroid cancer, 76 melanoma cancer, 148 colorectal cancer, 68 kidney cancer, 49 lymphoma, 67 pancreatic cancer, 74 liver & bile duct cancer, 140 gastric cancer, 20 larynx cancer, 26 pharynx cancer, 283 oral cancer, 72 esophageal cancer, 61 prostate cancer, 16 bladder cancer, 21 brain & CNS cancer, 18 multiple myeloma, 9 anus cancer, 4 testicular cancer, 5 vulva cancer, 18 penile cancer, 2 vagina cancer, 18 gallbladder cancer, 23 sarcoma cancer, 14 germ cell tumor, 8 squamous cell carcinoma, 6 unknown primary, 1957 normal control and 8 samples in the category of ‘others’ in test set TABLE-3. A set of 1957 normal samples were also kept in test set to test the accuracy of applying first cancer versus normal model and then applying TOOAI model to distinguish between multiple cancers. A multivariate classifier was derived into the training sets and evaluated in the testing sets. The TOOAI model gave 18 scores to each sample corresponding to endometrial cancer score, breast cancer score, cervical cancer score, ovarian cancer score, lung cancer score, leukemia cancer score, thyroid cancer score, melanoma cancer score, colorectal cancer score, kidney cancer score, lymphoma cancer score, pancreatic cancer score, liver & bile duct cancer score, gastric cancer score, head & neck cancer score, esophageal cancer, prostate cancer score and ‘others’ cancer score.

To test the accuracy of the double class prediction obtained from the TOOAI model for Endometrial cancer a confusion matrix with predicted and true label was generated based on using the Prediction probability score for the endometrial cancer which ultimately leads to distinction of endometrial cancer candidate from the others with 93.3% (See e.g., FIG. 10, TABLE-4).

To test the accuracy of the double class prediction obtained from the TOOAI model for Breast cancer a confusion matrix with predicted and true label was generated based on using the Prediction probability score for the Breast cancer which ultimately leads to distinction of breast cancer candidate from the others with 94.1% (See e.g., FIG. 10, TABLE-4).

To test the accuracy of the double class prediction obtained from the TOOAI model for Cervical cancer a confusion matrix with predicted and true label was generated based on using the Prediction probability score for the Cervical cancer which ultimately leads to distinction of cervical cancer candidate from the others with 96% (See e.g., FIG. 10, TABLE-4).

To test the accuracy of the double class prediction obtained from the TOOAI model for Ovarian cancer a confusion matrix with predicted and true label was generated based on using the Prediction probability score for the Ovarian cancer which ultimately leads to distinction of Ovarian cancer candidate from the others with 90% (See e.g., FIG. 10, TABLE-4).

To test the accuracy of the double class prediction obtained from the TOOAI model for Lung cancer a confusion matrix with predicted and true label was generated based on using the Prediction probability score for the Lung cancer which ultimately leads to distinction of lung cancer candidate from the others with 95.4% (See e.g., FIG. 10, TABLE-4).

To test the accuracy of the double class prediction obtained from the TOOAI model for leukemia a confusion matrix with predicted and true label was generated based on using the Prediction probability score for the leukemia cancer which ultimately leads to distinction of leukemia candidate from the others with 91.1% (See e.g., FIG. 10, TABLE-4).

To test the accuracy of the double class prediction obtained from the TOOAI model for Thyroid cancer a confusion matrix with predicted and true label was generated based on using the Prediction probability score for the Thyroid cancer which ultimately leads to distinction of Thyroid cancer candidate from the others with 90% (See e.g., FIG. 10, TABLE-4).

To test the accuracy of the double class prediction obtained from the TOOAI model for Melanoma a confusion matrix with predicted and true label was generated based on using the Prediction probability score for the Melanoma which ultimately leads to distinction of Melanoma cancer candidate from the others with 94.7% (See e.g., FIG. 10, TABLE-4).

To test the accuracy of the double class prediction obtained from the TOOAI model for Colorectal cancer a confusion matrix with predicted and true label was generated based on using the Prediction probability score for the Colorectal cancer which ultimately leads to distinction of Colorectal cancer candidate from the others with 95.2% (See e.g., FIG. 10, TABLE-4).

To test the accuracy of the double class prediction obtained from the TOOAI model for Kidney cancer a confusion matrix with predicted and true label was generated based on using the Prediction probability score for the Kidney cancer which ultimately leads to distinction of Kidney cancer candidate from the others with 86% (See e.g., FIG. 10, TABLE-4).

To test the accuracy of the double class prediction obtained from the TOOAI model for lymphoma a confusion matrix with predicted and true label was generated based on using the Prediction probability score for the lymphoma cancer which ultimately leads to distinction of non-Hodgkin's lymphoma candidate from the others with 89.7% (See e.g., FIG. 10, TABLE-4).

To test the accuracy of the double class prediction obtained from the TOOAI model for Pancreatic cancer a confusion matrix with predicted and true label was generated based on using the Prediction probability score for the endometrial cancer which ultimately leads to distinction of Pancreatic cancer candidate from the others with 98.5% (See e.g., FIG. 10, TABLE-4).

To test the accuracy of the double class prediction obtained from the TOOAI model for Liver cancer a confusion matrix with predicted and true label was generated based on using the Prediction probability score for the Liver cancer which ultimately leads to distinction of Liver cancer candidate from the others with 91.89% (See e.g., FIG. 10, TABLE-4).

To test the accuracy of the double class prediction obtained from the TOOAI model for Gastric cancer a confusion matrix with predicted and true label was generated based on using the Prediction probability score for the Gastric cancer which ultimately leads to distinction of Gastric cancer candidate from the others with 92.85% (See e.g., FIG. 10, TABLE-4).

To test the accuracy of the double class prediction obtained from the TOOAI model for Head & neck cancer a confusion matrix with predicted and true label was generated based on using the Prediction probability score for the endometrial cancer which ultimately leads to distinction of Head & neck cancer candidate from the others with 94.69% (See e.g., FIG. 10, TABLE-4).

To test the accuracy of the double class prediction obtained from the TOOAI model for esophageal cancer a confusion matrix with predicted and true label was generated based on using the Prediction probability score for the esophageal cancer which ultimately leads to distinction of esophageal cancer candidate from the others with 95.83% (See e.g., FIG. 10, TABLE-4).

To test the accuracy of the double class prediction obtained from the TOOAI model for prostate cancer a confusion matrix with predicted and true label was generated based on using the Prediction probability score for the prostate cancer which ultimately leads to distinction of prostate cancer candidate from the others with 98.3% (See e.g., FIG. 10, TABLE-4).

To test the accuracy of the double class prediction obtained from the TOOAI model for “others” cancer a confusion matrix with predicted and true label was generated based on using the Prediction probability score for the “others” cancer which ultimately leads to distinction of “others” cancer candidate from the others with 94.4% (See e.g., FIG. 10, TABLE-4).

Hence, as explained above, the system 100 and related method 200 may efficiently detect and distinguish cancer samples from the normal controls using a first CDAI Model, and further may efficiently detect and distinguish each individual cancer sample from the other cancer samples by using the TOOAI Model on samples identified as cancer-positive by the CDAI Model.

Following are the explanation of the exemplary processes and devices that may be used in the system 100 for executing the metabolomics process, and that were used in the present study conducted.

### Subjects and Methods

Serum samples were obtained either from biobanks in US and Europe or collected from various clinical sites/hospitals in India. The demographic and ethnic distribution of the specimens were shown in Table-1. Controls and disease cases were catalogued according to age-group, BMI, ethnicity and stages of cancer. All diagnoses were made in accordance with uniform histological and pathological guidelines.

### Serum Specimens

Blood samples were collected and processed according to standardized protocols. Each sample was assigned a unique laboratory identification number, which specified the order of processing and blinded laboratory personnel to sample identity. Samples were stored at −80 C until use.

### Sample Preparation

Metabolite extraction from serum was performed as explained previously. Briefly, all the serum samples were thawed on ice and mixed properly. 10 μl of each serum sample was taken in microfuge tube (1.5 ml), (Genaxy, Cat No. GEN-MT-150-C. S) and then 30 μl of chilled Methanol, (Merck, Cat.No. 1.06018.1000) to the sample, vortexed briefly and then kept at −20° C. for 60 minutes.

The sample was then centrifuged (Sorvall Legend Micro17, Thermo Fisher Scientific, Cat.No. Ligend Micro 17) at 10000 rpm for 10 minutes. After centrifugation 27 ul supernatant was collected in separate microfuge tube without disturbing the pellet and dried using Speed Vacuum, (ThermoFisher Scientific, Cat.No. SPD1030-230) at low energy for 30-35 minutes. Samples pellets were then re-suspended using 50 ul methanol:water (1:1, water:methanol) mixture for injection. Or the samples can be stored at −20° C. without re-suspending it.

Or

10 μl of each serum sample was taken in microfuge tube (1.5 ml), (Genaxy, Cat No. GEN-MT-150-C. S) and then 30 μl of chilled Methanol, (Merck, Cat.No. 1.06018.1000) to the sample, vortexed briefly and kept at −80° C. for 15 minutes. The sample was then centrifuged (Sorvall Legend Micro17, Thermo fisher Scientific, Cat.No. Ligend Micro 17) at 10000 rpm for 10 minutes. After centrifugation 27 ul supernatant was collected in separate microfuge tube without disturbing the pellet and dried using Speed Vacuum, (Thermo fisher Scientific, Cat. No. SPD1030-230) at low energy for 30-35 Minutes. Samples pellets were then re-suspended using 50 ul methanol:water (1:1, water:methanol) mixture for injection. Or the samples can be stored at −20° C. without re-suspending it.

Or

20 μl of each serum sample was taken in microfuge tube (1.5 ml), (Genaxy, Cat No. GEN-MT-150-C. S) and then 40 μl of chilled Methanol, (Merck, Cat.No. 1.06018.1000) to the sample, vortexed briefly and 200 ul of MTBE (Methyl Tertiarybutyl Ether) Cat.No. 306975-1 L was added to the sample tube and kept for 1 hr at room temp on shaker. 50 ul of water was added and vortexed briefly. The sample was then centrifuged (Sorvall Legend Micro17, Thermo fisher Scientific, Cat.No. Ligend Micro 17) at 3000 g for 10 minutes. After centrifugation, organic and aqueous phases were formed, with utmost care each phase was collected in a separate microfuge tube without disturbing the pellet and interface. 25 ul of each phase was collected and added in a fresh microfuge tube and dried using speed vacuum at low energy for 25-30 minutes (Thermo fisher Scientific, Cat. No. SPD1030-230) Samples pellets were then re-suspended using 50 ul methanol:water (1:1, water:methanol) mixture for injection. Or the samples can be stored at −20° C. without re-suspending it.

Or

10 μl of each serum sample was taken in microfuge tube (1.5 ml), (Genaxy, Cat No. GEN-MT-150-C. S) and then 400 μl of chilled Chloroform/Methanol/Water (1:3:1), (Merck, Cat.No. 1.06018.1000, Merck, Cat.No.C2432-1L, Merck, Cat. No 1.15333.1000) to the sample, and vortexed for 1 min. The sample was then centrifuged (Sorvall Legend Micro17, Thermo fisher Scientific, Cat. No. Ligend Micro 17) at 13000 g for 3 minutes. After centrifugation, 80 ul of supernatant was collected in a separate microfuge tube without disturbing the pellet and dried using speed vacuum at low energy for 25-30 minutes (Thermo fisher Scientific, Cat. No. SPD1030-230) Samples pellets were then re-suspended using 50 ul methanol:water (1:1, water:methanol) mixture for injection. Or the samples can be stored at −20° C. without re-suspending it.

### LC-MS/MS Analysis

Untargeted LC-MS/MS metabolomics experiments were performed using Dionex LC system (Ultimate 3000) coupled online with QExactive Plus (Thermo Scientific). Each extracted metabolite sample was injected (10 ul for positive ESI ionization) onto Acquity UPLC HSS T3 from Waters (1.8 micron, dimensions—2.1×100 mm, Part No. 186003539), which was heated to 40 C. The flow rate was 0.3 ml/min. Mobile phase A was (water+0.1% formic acid), and mobile phase B was (methanol+0.1% formic acid). The mobile phase was kept isocratic at 5% B for 1 min, and was increased to 95% B in 7 min and kept for another two min at 95% B, the mobile phase composition returned to 5% B in 14 min. The ESI voltage was 4 kV. The mass accuracy of QExactive mass spectrometry was less than 5 ppm and calibrated at recommended schedule prior to each batch run. The mass scan range is from 66.7-1000 Da, and resolution was set to 35000. The maximum inject time for orbitrap was 100 msec while, AGC target was optimized with 1e6.

Optimization and validation of Liquid chromatography and mass spectrometry methods To obtain the reliable and consistent outcome of serum metabolite profile from the mass spectrometry, we have optimized several parameters to counter the faulty data recording. Out of many steps taken into account, our primary focus was on the matching chromatogram profile as well as on the quality of data obtained each time a sample is run. We have called these steps Quality checks (QCs). We have designated 03 major QCs and detailed description is provided below.

Step QC1 of System 114 involves Chromatogram profile matching. A faulty chromatogram may be a result of faulty sample extraction, or due to an error in the mass spectrometry setting. These errors impact on the quality of data, which then compromise the overall prediction accuracies of the algorithms. A sequential neural network model was built to detect these faults based on variations in the chromatogram profiles. The chromatogram obtained from the mass spectrometer for each sample were first converted into jpeg format. The image was then scaled to appropriate width and length for the model training. The Image was binarized in order to segregate the chromatogram, which facilitates more efficient analysis. Keras Sequential neural network model was then used to train-test validate the model. We employed three Keras Conv2D 2D Convolution Layers, which create a convolution kernel that is wound with layered inputs that help produce a tensor of outputs. An 80-20 train-test split was taken. Adam optimizer was subsequently used to update network weights iteratively based on the training data. The threshold used for the correct detection was 0.5 and samples which showed a Quality Control (QC) score of <0.5 were designated as having passed QC1. Samples with >0.5 QC score were rejected as having failed at the QC1 step. We obtained 100% detection accuracy for faulty chromatograms as shown in FIG. 6 A.

Step QC2 of System 114 monitors for the presence of critical m/z ions. The second step of System 114, which is called QC2, monitored for the presence of at least 6 out of 9 critical ions with m/z values ranging from 100 to 800. The distribution of intensity and RT of these 9 ions is shown in the FIG. 6 B. Presence of 6 or more of the 9 critical ions in the chromatogram of a sample is taken as the threshold criteria for passing the QC2 Step. Samples with <6 out of the 9 critical masses are rejected as having failed the QC2 step. The QC2 step is important for the accurate identification of cancer samples because those samples that do not pass this step have a higher likelihood of misclassification by the CDAI algorithm (FIG. 6 B).

Step QC3 of System 114 monitors matrix occupancy: Another layer of quality check that was introduced along with previous two quality checks involved an assessment of matrix occupancy. This layer is Step QC3 and it relies on the percentage of features that matches with the matrix size. The threshold was optimized with multiple runs of poor-quality samples, or with improper mass spectrometry run conditions. Based on these studies the threshold for minimum matrix occupancy was set at 15%. This threshold was confirmed through multiple validation exercises, which was then found to improve the robustness and accuracy of prediction CDAI algorithm. With the trained model for QC3, faulty samples could be captured with 100% accuracy as shown in FIG. 6 C.

### Results

Regarding frequency matching variables including age, race, BMI, and cancer stages, the demographic and ethnicity distribution (Table-1) for controls and cancer patients was balanced. In the training or testing sets, none of the observed variations in the distribution of these variables between control and illness cases attained statistical significance. Approximately 80% of the cancer cases came from stage I. FIG. 2 shows a schematic of the complete procedure, with illustrations of the key steps in each step. The Dionex LC system connected online with the QExactive Plus mass spectrometer received injections of the isolated metabolites from the serum. The preprocessing of the data is initially depicted schematically in FIG. 2. The following list includes the various data preprocessing steps:

1. Extracting metabolic feature nodes:
Data from metabolomics are known to contain mass inaccuracies. As a result, the mass of the same identified metabolite in several samples will vary slightly. This makes it difficult to compare the intensity of the same metabolite across samples affecting downstream AI-based analysis which requires this intensity comparison on the grounds of robust feature intensity values as well as dimensionality. To overcome this, we employed various simultaneous and parallel approaches. The metabolic feature nodes were identified using fixed mass boxes covering the entire mass range. The thresholds for these mass boxes were defined using different technique like mass KNN clustering, uniform linear separation, virtual lock mass (VLM) based strategy. Upon exhaustive search of techniques over numerous samples in combination with HMDB databases we were able to identify robust mass ranges, and these were used to align any new dataset.
2. Data filtering: The presence of noise in a data set can increase the model complexity and time of learning which degrades the performance of learning algorithms. Data filtering is a process of noise reduction as well as dimensionality reduction by which an initial set of raw data contains target specific attributes and is reduced to more manageable data format.
3. Data Normalization/standardization: Normalization techniques are required to reduce the variations in the data since the metabolic data fluctuates under different mass spectrometer parameters. Different normalization methods were tried such as Quantile Normalization, Variance Stabilization Normalization, Best Normalization, Probabilistic Quotient Normalization.
   Data standardization is a data processing workflow that converts the structure of different datasets into one common format of data. It deals with the transformation of datasets after the data are collected from different sources and before it is loaded into target systems. Various Data standardization methods like standard normalization, L1 and L2 norm standardization were employed in the data set
   A combination of Standardization and Normalization was used for the two-tiered algorithm. This method was further adapted to our datasets to enable the normalization of new samples with respect to training datasets and testing one sample at a time.
4. Missing value imputation: It is well established that missing values in untargeted metabolomics data can be troublesome. In large metabolite panels, measurement values are frequently missing and, if neglected or sub-optimally imputed, can cause biased study results. Various supervised and unsupervised multiple imputation techniques like Iterative Imputer, missforest, simple impute, KNN impute were employed and the effects of sample size, percentage missing, and correlation structure on the accuracy of the imputation methods were evaluated.
5. Feature reduction: Dimensionality reduction is the process of reducing the number of random variables under consideration, by obtaining a set of principal variables. This is a critical step in high dimensional data as it takes care of curse of dimensionality, Multi-collinearity, Noise, computational cost, and Visualization. Feature Extraction can be Unsupervised (PCA) or supervised (LDA, PLS-DA etc.). Various Feature reduction techniques were evaluated based on data variance capture and class separation namely PLSDA R2 maximization, RFE, PCA, Non-negative Matrix Factorization, LDA.
6. Machine learning model development: After going the above pipeline the data is fed into the AI machinery. AI models were made to differentiate cancers from normal and then between the individual cancers.

Keeping in mind clinical applications of the AI model in the present invention, a tiered approach was used here in which first, an AI model was developed for cancer signal detection (the CDAI Model) and then next model, the TOOAI Model, classifies the tissue of origin for the cancer positive sample. Out of total 8971 samples, endometrial cancer (n=445), breast cancer (n=652), cervical cancer (n=458), ovarian cancer (n=488), lung cancer (n=307), leukemia (n=157), thyroid cancer (n=169), melanoma (n=151), colorectal cancer (n=296), kidney cancer (n=136), lymphoma (n=97), pancreatic cancer (n=134), liver & bile duct cancer (n=147), gastric cancer (n=279), head & neck cancer (n=638), esophageal cancer (n=143) and prostate cancer (n=122) and other cancers (n=254). and 3914 normal control samples. The matrix produced above was utilized to examine whether there are any differences between these samples based on metabolic data. The 18 cancer classes and normal controls were used to create a PLS DA plot, as seen in FIG. 7. The graphic unmistakably demonstrates how cancer samples may be differentiated from normal control samples using their metabolic characteristics. An AI analysis was performed on the data as detailed below to uncover common patterns in metabolite fluctuations within cancer samples, which is distinct from normal control samples, in order to measure how well these can be distinguished.

Development of the algorithm for the CDAI Model Out of the total 8971 cancer samples, 5057 samples were from the thirty-two (32) cancer classes mentioned in TABLE-1 and 3914 were normal controls. Normal controls were samples from volunteers having no cancer. The data was randomly partitioned into training and test datasets in equal proportion. This resulted in 2479 Cancer samples and 1957 Controls in training set, and 2594 Cancer samples and 1957 Controls in test set (TABLE-3). Complete schematic of the steps for cancer detection is shown in FIG. 1 and model was evaluated using parameters log loss, Accuracy, Sensitivity, Specificity.

Parametric machine learning model were applied on the training data to obtain a score function depending on the intensity values of the features. The Class balancing parameters were configured in the model to deal with the imbalance of Cancer and the control samples the training dataset. The final trained model used to evaluate the score of each sample using the following formulae:

$$y\_score = {x}_{0} + {x}_{{1}^{*}} {I}_{1} + {x}_{{2}^{*}} {I}_{2} + {x}_{{3}^{*}} {I}_{3} + … + {x}_{{n}^{*}} {I}_{n}$$

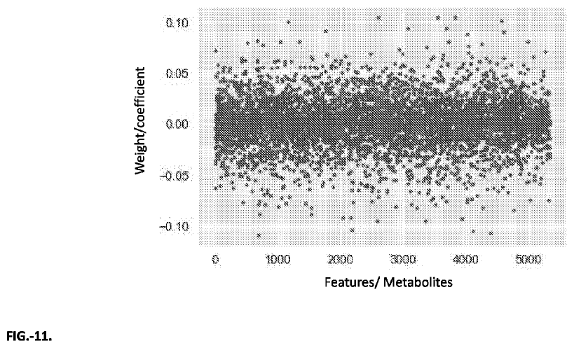

Here, x0 is a constant number, Iᵢ (1<=i<=n) is the intensity of metabolite i present in the respective sample. The total number of metabolites is represented by the symbol n(n∈[1000,8300]). FIG. 11 gives the value of coefficient xᵢ(1<=i<=n) for each metabolite.

The y score plot of the trained model as applied on test set for a single partition of data containing 32 cancer classes and normal control shown for example in FIG. 9 The scatter plot shows the Model Score for Controls and Cancer cases. The model scores are clearly seen to be different between Controls and Cancer samples where on applying a threshold of y-score of zero to differentiate between two types of results in a confusion matrix as shown. Sensitivity, Specificity, and Accuracy can be calculated from the below formulae:

$$\begin{matrix}Accuracy \text{:} \frac{TP + TN}{TP + TN + FP + FN} \\ Sensitivity \text{:} \frac{TP}{TP + FN} \\ Specifity \text{:} \frac{TN}{TN + FP}\end{matrix}$$

| | Predicted | | |
|---|---|---|---|
| | | Negative | Positive |
| Actual | Negative | True Negative (TN) | False Positive (FP) |
| | Positive | False Negative (FN) | True Positive (TP) |

This results in Accuracy of 99.8%, Sensitivity of 99.26% and Specificity of 99.64%. (FIG. 9)

### Development of the Algorithm for the TOOAI Model

To advance on the clinical manifestation of the cancer positive samples i.e., the cancer tissue of origin of the cancer signal detected in the CDAI model, we developed the TOOAI model.

In brief the TOOAI model is a multiclass algorithm that evaluates the probability score for the cancer positive sample suggesting the tissue from which the cancer positive signal has originated.

For developing the algorithm for tissue of origin (TOOAI Model) the dataset containing the cancer samples were first processed according to the steps explained in the earlier section. Here, out of total 5057 Cancer samples, samples were Endometrial Cancer, Breast Cancer, Cervical Cancer, Ovarian Cancer, Lung Cancer, Kidney Cancer, Thyroid cancer, Acute myeloid lymphoma, non-Hodgkin's lymphoma, Pancreatic cancer, Colorectal cancer, Liver cancer, Gastric cancer, Melanoma cancer, head & neck cancer, esophageal cancer, prostate cancer and ‘others’ TABLE-2. The data was randomly partitioned into training and test datasets in equal proportion and complete distribution of training and testing distribution in this layer is shown in TABLE-3.

The Machine learning environment were set for python 3.10.4. Various algorithms were used to obtain the predict probability function for the cancer samples, where each probability score suggests the occurrence of that cancer type. To accomplish this, we used Support vector machine, Logistic one versus rest, Stochastic gradient descent algorithms. The optimal set of hyperparameters for these parameters were obtained using exhaustive training testing by python Grid search CV package. This resulted in 18 probability scores for each sample, with each score defining probability of the respective sample belonging to one of the 18 cancer tissue type. The trained algorithm finds tissue of origin probability for each of the sample according to the formulae below:

$$p ( Endometrial ) = \frac{1}{1 + {e}^{y 0 + y 1 * I 1 + y 2 + I 2 + ⋯}}$$

$$p ( Breast ) = \frac{1}{1 + {e}^{a 0 + y 1 * I 1 + y 2 + I 2 + ⋯}}$$

$$P ( Cervical ) = \frac{1}{1 + {e}^{a 1 + y 1 * I 1 + y 2 + I 2 + ⋯}}$$

$$P ( Ovarian ) = \frac{1}{1 + {e}^{a 2 + y 1 * I 1 + y 2 + I 2 + ⋯}}$$

$$P ( Thyroid ) = \frac{1}{1 + {e}^{a 3 + y 1 * I 1 + y 2 + I 2 + ⋯}}$$

Here, a₀, a₁, a₂, . . . , aₙ are constant number, Iᵢ (I<=i<=8000) is the Normalized intensity of metabolite i present in the respective sample. N is number of cancer type classes included in the training set.

The final model having the highest double class prediction accuracy in the test set was chosen for further evaluation, here the double class prediction accuracy will mean an occurrence of correct prediction in the top two prediction from the model using the above defined probability function.

The Double class prediction accuracies were evaluated for the single test dataset as an example and the confusion matrix for the final prediction are shown in FIG. 10. The table 4 shows double class prediction accuracy for the same. The prediction accuracy for the double class prediction from the model were evaluated using the following formulae:

$$Accuracy = \begin{matrix}\frac{\begin{matrix}Total correctly predicted sample \\ ( True prediction ⋂ Prediction ( 1 , 2 ) ∈ \\ max ( P ( breast ) , P ( Uterine ) , … , P ( N ) )\end{matrix}}{Total number of sample in Cancer subclass} \\ \end{matrix}$$

Ranking cancer specific features: The feature derived for the model prediction involves metabolites from the HMDB database. Feature ranking help us identify the key metabolites that are contributing to the model accuracy, also broaden the scope of prediction done by the model in sense of molecular translation of cancer signature obtained. Various Feature ranking methods parametric, non-parametric based approaches were used and the top 100 metabolites obtained for Cancer signal detection step relevant for all the cancer type were obtained shown in TABLE-5.

TABLE 2
Distribution of samples in TOOAI model w.r.t cancer stages

| | 0 | I | II | III | IV |
|---|---|---|---|---|---|
| Normal control (n = 3914) | 0 | 0 | 0 | 0 | 0 |
| Endometrial cancer (n = 445) | 0 | 350 | 64 | 23 | 8 |
| Breast cancer (n = 652) | 36 | 335 | 179 | 80 | 22 |
| Cervical cancer (n = 458) | 70 | 236 | 92 | 43 | 17 |
| Ovarian cancer (n = 488) | 19 | 292 | 96 | 59 | 22 |
| Lung cancer (n = 307) | 7 | 112 | 55 | 80 | 53 |
| Leukemia (n = 157) | 0 | 10 | 15 | 29 | 32 |
| Thyroid cancer (n = 169) | 0 | 83 | 49 | 32 | 5 |
| Melanoma (n = 151) | 2 | 82 | 42 | 18 | 7 |
| Colorectal cancer (n = 296) | 0 | 82 | 100 | 95 | 19 |
| Kidney cancer (n = 136) | 0 | 77 | 30 | 21 | 8 |
| Lymphoma (n = 97) | 0 | 13 | 15 | 9 | 10 |
| Pancreatic cancer (n = 134) | 0 | 32 | 77 | 17 | 8 |
| Liver & bile duct cancer (n = 147) | 0 | 62 | 41 | 32 | 12 |
| Gastric cancer (n = 279) | 0 | 103 | 110 | 57 | 9 |
| Head and neck cancer (n = 638) | 14 | 128 | 288 | 147 | 61 |
| Oesophageal cancer (n = 143) | 0 | 26 | 61 | 42 | 14 |
| Prostate cancer (n = 122) | 0 | 17 | 37 | 55 | 13 |
| Other cancers (n = 254) | 0 | 33 | 88 | 97 | 23 |

TABLE 3
Distribution of samples for training and testing

| | Total No. | | |
|---|---|---|---|
| Type of sample | of Samples | Training | Testing |
| Normal controls | 3914 | 1957 | 1957 |
| Endometrial cancer | 445 | 222 | 223 |
| Breast cancer | 652 | 326 | 326 |
| Cervical cancer | 458 | 229 | 229 |
| Ovarian cancer | 488 | 244 | 244 |
| Lung cancer | 307 | 153 | 154 |
| Leukemia | 157 | 78 | 79 |
| Thyroid cancer | 169 | 84 | 85 |
| Melanoma | 151 | 75 | 76 |
| Colorectal cancer | 296 | 148 | 148 |
| Kidney cancer | 136 | 68 | 68 |
| Lymphoma | 97 | 48 | 49 |
| Pancreatic cancer | 134 | 67 | 67 |
| Liver and bile duct cancer | 147 | 73 | 74 |
| Gastric cancer | 279 | 139 | 140 |
| Larynx cancer | 20 | 0 | 20 |
| Pharynx cancer | 52 | 26 | 26 |
| Oral cancer | 566 | 283 | 283 |
| Oesophageal cancer | 143 | 71 | 72 |
| Prostate cancer | 122 | 61 | 61 |
| Bladder cancer | 32 | 16 | 16 |
| Brain & CNS cancer | 42 | 21 | 21 |
| Multiple myeloma | 18 | 0 | 18 |
| Gall bladder cancer | 35 | 17 | 18 |
| Sarcoma | 45 | 22 | 23 |
| Testicular cancer | 4 | 0 | 4 |
| Vulval cancer | 5 | 0 | 5 |
| Anal cancer | 9 | 0 | 9 |
| Vaginal cancer | 2 | 0 | 2 |
| Penile cancer | 18 | 0 | 18 |
| Unknown primary cancer | 6 | 0 | 6 |
| Germ cell tumor | 14 | 0 | 14 |
| Squamous cell carcinoma | 8 | 0 | 8 |
| Other cancers | 16 | 8 | 8 |

TABLE 4
Tissue of origin (TOOAI Model) results

| Cancer Type | Samples correctly identified | Sensitivity (%) |
|---|---|---|
| Breast | 307/326 | 94 |
| Endometrial | 208/223 | 93.2 |
| Cervical | 220/229 | 96 |
| Ovarian | 222/244 | 90.9 |
| Lung | 147/154 | 95.4 |
| AML | 72/79 | 91 |
| Thyroid | 77/85 | 90.5 |
| Melanoma | 72/76 | 94.7 |
| Colorectal | 141/148 | 95.2 |
| Kidney | 59/68 | 86.7 |
| NHL | 44/49 | 89.8 |
| Pancreatic | 66/67 | 98.5 |
| Liver | 68/74 | 91.9 |
| Gastric | 130/140 | 92.8 |
| Head & neck | 268/283 | 94.6 |
| esophageal | 69/72 | 95.8 |
| Prostate | 60/61 | 98.3 |
| Others | 204/216 | 94.4 |

TABLE 5
List of top 100 metabolites

| | Metabolite names |
|---|---|
| 1 | (—)-Aspidospermine |
| 2 | (11E)-16-Hydroxy-3-methyl-1,7-dioxo-3,4,5,6,7,8,9,10-octahydro-1H-2- |
| | benzoxacyclotetradecin-14-yl hydrogen sulfate |
| 3 | (11R,23R)-14,17,20-Trihydroxy-14,20-dioxido-8,26-dioxo-9,13,15,19,21,25- |
| | hexaoxa-14lambda~5~,20lambda~5~-diphosphatritriacontane-11,23-diyl |
| | dioctanoate |
| 4 | (14R,20S)-17,20,21-Trihydroxy-2-methyl-17-oxido-11-oxo-12,16,18-trioxa- |
| | 17lambda~5~-phosphahenicosan-14-yl (9Z,11Z)-9,11-octadecadienoate |
| 5 | (14R,20S)-17,20,23,23-Tetrahydroxy-2-methyl-17,23-dioxido-11-oxo-12,16,18,22- |
| | tetraoxa-17lambda~5~,23lambda~5~-diphosphatricosan-14-yl (9Z,11Z)-9,11- |
| | octadecadienoate |
| 6 | (19R,25S)-22,25,26-Trihydroxy-22-oxido-16-oxo-17,21,23-trioxa-22lambda~5~- |
| | phosphahexacosan-19-yl (9Z)-9-hexadecenoate |
| 7 | (1E)-1-{[1-(Methyldisulfanyl)propyl]sulfinyl}-1-propene |
| 8 | (1E)-7-Hydroxy-5-(2-hydroxy-2-propanyl)-2-methyl-8-methylene-1-cyclodecen-1- |
| | yl acetate |
| 9 | (1E)-N-(1-Carboxy-2-hydroxypropyl)-3-[(carboxymethyl)amino]-5-hydroxy-5- |
| | (hydroxymethyl)-2-methoxy-2-cyclohexen-1-iminium |
| 10 | (1R,3R,4S,5R)-3,4-Dihydroxy-1,5-bis{[(2E)-3-(4-hydroxy-3-methoxyphenyl)-2- |
| | propenoyl]oxy}cyclohexanecarboxylic acid |
| 11 | (1R,3R,5R)-2-[(2S)-2-Amino-2-(3,5-dihydroxyadamantan-1-yl)acetyl]-2- |
| | azabicyclo[3.1.0]hexane-3-carbonitrile |
| 12 | (1R,7R,10R)-4,10,11,11-Tetramethyl-3-methylenetricyclo[5.3.1.0~1,5~]undec-4- |
| | ene |
| 13 | (1R,8E,9Z,14S,15S,17R)-8-Ethylidene-15-[(2R)-1-hydroxy-2-propanyl]-17-methyl- |
| | 3,11-dioxo-2,6,12-trioxatricyclo[12.2.1.0~4,9~]heptadeca-4,9-dien-7-yl beta-D- |
| | glucopyranoside |
| 14 | (1S,5R)-5-Isopropenyl-2-methyl-2-cyclohexen-1-yl beta-D-glucopyranoside |
| 15 | (1S,7R,7aS)-7-Acetoxy-1-[(4S,5E)-6-carboxy-4-hydroxy-5-hepten-2-yl]-5- |
| | {[(1S,4S)-3,3-dimethyl-2-oxo-7-oxabicyclo[2.2.1]hept-1-yl]methyl}-1,7a-dimethyl- |
| | 2,6,7,7a-tetrahydro-1H-indene-4-carboxylic acid |
| 16 | (1Z)-1-({1-[(1E)-1-Propen-1-yldisulfanyl]propyl}sulfanyl)-1-propene |
| 17 | (1Z)-1-Ethylidene-7-isopropyl-3-[(2-methylbutanoyl)oxy]-4-methylene-2- |
| | oxooctahydro-1H-inden-5-yl (2E)-3-methyl-2-pentenoate |
| 18 | (1Z)-N-{1-[(8Z,11E,13E)-10-sec-Butyl-8,11-dihydroxy-16-methoxy-2-oxa-6,9,12- |
| | triazatricyclo[13.3.1.0~3,7~]nonadeca-1(19),8,11,13,15,17-hexaen-6-yl]-4-methyl- |
| | 1-oxo-2-pentanyl}-2-(dimethylamino)-3-phenyl |
| | propanimidic acid |
| 19 | (1Z)-N-{1-[(8Z,11E,13E)-10-sec-Butyl-8,11-dihydroxy-16-methoxy-2-oxa-6,9,12- |
| | triazatricyclo[13.3.1.0~3,7~]nonadeca-1(19),8,11,13,15,17-hexaen-6-yl]-4-methyl- |
| | 1-oxo-2-pentanyl}-2-(methylamino)-3-phenylpropanimidic acid |
| 20 | (1aR,5aS,7aS,8aS,9aS,9dS)-5a,7a-Dimethyl-5′-oxoicosahydro-3′H- |
| | spiro[cyclopropa[4,5]cyclopenta[1,2-a]cyclopropa[l]phenanthrene-8,2′-furan]-3- |
| | sulfonic acid |
| 21 | (1xi)-1,5-Anhydro-1-[(2R,3R)-2-(3,4-dihydroxyphenyl)-3,5,7-trihydroxy-3,4- |
| | dihydro-2H-chromen-6-yl]-D-glucitol |
| 22 | (2,4-Dihydroxyphenyl)[6-(2,4-dihydroxyphenyl)-2-{4-[(E)-2-(2,4- |
| | dihydroxyphenyl)vinyl]-2,6-dihydroxyphenyl}-4-methyl-3-cyclohexen-1- |
| | yl]methanone |
| 23 | (2-Hydroxy-2-oxido-1,3,2-dioxaphospholan-4-yl)methyl (9Z)-9-octadecenoate |
| 24 | (21R,27S)-24,27,30,30-Tetrahydroxy-24,30-dioxido-18-oxo-19,23,25,29-tetraoxa- |
| | 24lambda~5~,30lambda~5~-diphosphatriacontan-21-yl (4Z,7Z,10Z,13Z,16Z)- |
| | 4,7,10,13,16-docosapentaenoate |
| 25 | (21R,27S)-24,27,30,30-Tetrahydroxy-24,30-dioxido-18-oxo-19,23,25,29-tetraoxa- |
| | 24lambda~5~,30lambda~5~-diphosphatriacontan-21-yl (7Z,10Z,13Z,16Z)- |
| | 7,10,13,16-docosatetraenoate |
| 26 | (22E)-3,5,9,14-Tetrahydroxyergosta-7,22-dien-6-one |
| 27 | (24E)-3,15,22-Triacetoxy-7-methoxylanosta-8,24-dien-26-oic acid |
| 28 | (24Z)-3-Acetoxy-15-hydroxy-7-methoxylanosta-8,24-dien-26-oic acid |
| 29 | (2E)-1-(3-{6-[2,4-Dihydroxy-3-(3-methyl-2-buten-1-yl)benzoyl]-5-(4- |
| | hydroxyphenyl)-3-methyl-2-cyclohexen-1-yl}-2,4-dihydroxyphenyl)-3-(4- |
| | hydroxyphenyl)-2-propen-1-one |
| 30 | (2E)-2-Tetradecenal |
| 31 | (2E)-3-(2-Furyl)-2-phenylacrylaldehyde |
| 32 | (2E)-3-(3,4-Dimethoxyphenyl)-N-[2-(3-hydroxy-4- |
| | methoxyphenyl)ethyl]acrylamide |
| 33 | (2E)-3-(4-Hydroxy-3-methoxyphenyl)-N-(4-oxobutyl)acrylamide |
| 34 | (2E)-3-(6,7-Dimethoxy-1,3-benzodioxol-5-yl)-2-propen-1-yl hydrogen sulfate |
| 35 | (2E)-3-[4-(Sulfooxy)phenyl]acrylic acid |
| 36 | (2E)-4-{2-(2,4-Dihydroxyphenyl)-3-[(2Z)-3,7-dimethyl-2,6-octadien-1-yl]-5,7- |
| | dihydroxy-4-oxo-3,4-dihydro-2H-chromen-6-yl}-2-methyl-2-buten-1-yl hydrogen |
| | sulfate |
| 37 | (2E)-hexadecenoylcarnitine |
| 38 | (2E,4E)-5-[1-Hydroxy-2-(hydroxymethyl)-6,6-dimethyl-4-oxo-2-cyclohexen-1-yl]- |
| | 3-methyl-2,4-pentadienoic acid |
| 39 | (2E,4Z)-5-(4-Hydroxyphenyl)-1-(1-piperidinyl)-2,4-pentadien-1-one |
| 40 | (2R)-1-(Octanoyloxy)-3-(phosphonooxy)-2-propanyl myristate |
| 41 | (2R)-1-(Pentadecanoyloxy)-3-(phosphonooxy)-2-propanyl docosanoate |
| 42 | (2R)-1-(Phosphonooxy)-3-(tetradecanoyloxy)-2-propanyl (6Z,9Z,12Z)-6,9,12- |
| | octadecatrienoate |
| 43 | (2R)-1-[(5Z,8Z,11Z,14Z)-5,8,11,14-Icosatetraen-1-yloxy]-3-(phosphonooxy)-2- |
| | propanyl acetate |
| 44 | (2R)-1-[(5Z,8Z,11Z,14Z)-5,8,11,14-Icosatetraenoyloxy]-3-(phosphonooxy)-2- |
| | propanyl (4Z,7Z,10Z,13Z,16Z,19Z)-4,7,10,13,16,19-docosahexaenoate |
| 45 | (2R)-1-[(6Z,9Z,12Z)-6,9,12-Octadecatrienoyloxy]-3-(phosphonooxy)-2-propanyl |
| | (5Z,8Z,11Z,14Z,17Z)-5,8,11,14,17-icosapentaenoate |
| 46 | (2R)-1-[(6Z,9Z,12Z,15Z)-6,9,12,15-Octadecatetraenoyloxy]-3-(phosphonooxy)-2- |
| | propanyl (4Z,7Z,10Z,13Z,16Z,19Z)-4,7,10,13,16,19-docosahexaenoate |
| 47 | (2R)-1-[(Hydroxy{[(1S,5R)-2,3,4,6-tetrahydroxy-5- |
| | (phosphonooxy)cyclohexyl]oxy}phosphoryl)oxy]-3-[(9Z,12Z)-9,12- |
| | octadecadienoyloxy]-2-propanyl (11Z)-11-icosenoate |
| 48 | (2R)-1-[(Hydroxy{[(2R,3R,5S,6R)-2,3,4,5,6- |
| | pentahydroxycyclohexyl]oxy}phosphoryl)oxy]-3-(palmitoyloxy)-2-propanyl |
| | stearate |
| 49 | (2R)-1-{[(2-Aminoethoxy)(hydroxy)phosphoryl]oxy}-3-hydroxy-2-propanyl (13Z)- |
| | 13-docosenoate |
| 50 | (2R)-1-{[(2-Aminoethoxy)(hydroxy)phosphoryl]oxy}-3-hydroxy-2-propanyl |
| | (4Z,7Z,10Z,13Z,16Z)-4,7,10,13,16-docosapentaenoate |
| 51 | (2R)-1-{[(2-Aminoethoxy)(hydroxy)phosphoryl]oxy}-3-hydroxy-2-propanyl |
| | docosanoate |
| 52 | (2R)-2-(Octanoyloxy)-3-(phosphonooxy)propyl icosanoate |
| 53 | (2R)-2-(Pentadecanoyloxy)-3-(phosphonooxy)propyl (9Z)-9-hexadecenoate |
| 54 | (2R)-2-Hydroxy-3-(phosphonooxy)propyl (11Z,14E)-11,14-icosadienoate |
| 55 | (2R)-2-Hydroxy-3-(phosphonooxy)propyl (13Z,16Z)-13,16-docosadienoate |
| 56 | (2R)-2-Hydroxy-3-(phosphonooxy)propyl (6Z,9Z,12Z,15Z)-6,9,12,15- |
| | octadecatetraenoate |
| 57 | (2R)-2-Hydroxy-3-(phosphonooxy)propyl (8Z,11Z,14Z)-8,11,14-icosatrienoate |
| 58 | (2R)-2-[(5Z,8Z,11Z,14Z,17Z)-5,8,11,14,17-Icosapentaenoyloxy]-3-[(6Z,9Z,12Z)- |
| | 6,9,12-octadecatrienoyloxy]propyl 2-(trimethylammonio)ethyl phosphate |
| 59 | (2R)-3-(Phosphonooxy)-2-[(9Z)-9-tetradecenoyloxy]propyl pentadecanoate |
| 60 | (2R)-3-({[(2S)-2,3-Dihydroxypropoxy](hydroxy)phosphoryl}oxy)-2-[(10- |
| | methyldodecanoyl)oxy]propyl 10-methyldodecanoate |
| 61 | (2R)-3-[(1Z)-1-Hexadecen-1-yloxy]-2-hydroxypropyl dihydrogen phosphate |
| 62 | (2R)-3-{[(2-Aminoethoxy)(hydroxy)phosphoryl]oxy}-2-hydroxypropyl (9Z)-9- |
| | hexadecenoate |
| 63 | (2R,3R,4R,5S)-6-{[(8-Chloro-1-methyl-1,2,4,5-tetrahydro-3H-3-benzazepin-3- |
| | yl)carbonyl]oxy}-3,4,5-trimethyltetrahydro-2H-pyran-2-carboxylic acid |
| 64 | (2R,3S)-3-(4,7-Nonadienoyl)-2-oxiranecarboxamide |
| 65 | (2R,3S)-3-Hydroxy-8-methyl-8-azabicyclo[3.2.1]octane-2-carboxylic acid |
| 66 | (2R,3S,4S,5R)-2-(6-hydroxy-2-imino-3,7-dihydropurin-9-yl)-5- |
| | (hydroxymethyl)tetrahydrofuran-3,4-diol |
| 67 | (2R,8S)-2-[(9Z)-9-Hexadecenoyloxy]-5,8,11,11-tetrahydroxy-5,11-dioxido-4,6,10- |
| | trioxa-5lambda~5~,11lambda~5~-diphosphaundec-1-yl (9Z,12Z,15Z)-9,12,15- |
| | octadecatrienoate |
| 68 | (2S)-1-Hydroxy-3-[(6Z,9Z,12Z,15Z)-6,9,12,15-octadecatetraenoyloxy]-2-propanyl |
| | (5Z,8Z,11Z,14Z,17Z)-5,8,11,14,17-icosapentaenoate |
| 69 | (2S)-1-Hydroxy-3-[(9Z)-9-tetradecenoyloxy]-2-propanyl |
| | (4Z,7Z,10Z,13Z,16Z,19Z)-4,7,10,13,16,19-docosahexaenoate |
| 70 | (2S)-1-[(7Z,10Z,13Z,16Z)-7,10,13,16-Docosatetraenoyloxy]-3-hydroxy-2-propanyl |
| | (7Z,10Z,13Z,16Z,19Z)-7,10,13,16,19-docosapentaenoate |
| 71 | (2S)-2-(Docosanoyloxy)-3-(pentadecanoyloxy)propyl (4Z,7Z,10Z,13Z,16Z,19Z)- |
| | 4,7,10,13,16,19-docosahexaenoate |
| 72 | (2S)-2-Amino-5-({(2R)-3-[(2-amino-2-carboxyethyl)disulfanyl]-1- |
| | [(carboxymethyl)amino]-1-oxo-2-propanyl}amino)-5-oxopentanoic acid (non- |
| | preferred name) |
| 73 | (2S)-2-[(3E,7E,11E)-13-Hydroxy-4,8,12-trimethyl-3,7,11-tridecatrien-1-yl]-2,7,8- |
| | trimethyl-6-chromanol |
| 74 | (2S)-2-{[9-(3,4-Dimethyl-5-propyl-2-furyl)nonanoyl]oxy}-3-hydroxypropyl 11- |
| | (3,4-dimethyl-5-propyl-2-furyl)undecanoate |
| 75 | (2S)-3-Hydroxy-2-(octanoyloxy)propyl decanoate |
| 76 | (2S)-3-Phenyl-2-({[(3S,4S,5R)-2,3,4-trihydroxy-5-(hydroxymethyl)tetrahydro-2- |
| | furanyl]methyl}amino)propanoic acid (non-preferred name) |
| 77 | (2S)-6-Amino-2-[(E)-(hydroxymethylene)amino]hexanimidic acid |
| 78 | (2S,3R,4E)-2-(Dodecanoylamino)-3-hydroxy-4-octadecen-1-yl 2- |
| | (trimethylammonio)ethyl phosphate |
| 79 | (2S,3R,4S,5S,6S)-6-(4-{4-[(2R)-2-Amino-2-carboxyethyl]phenoxy}-2,6- |
| | diiodophenoxy)-3,4,5-trihydroxytetrahydro-2H-pyran-2-carboxylic acid |
| 80 | (2Z)-12-Hydroxy-2-(hydroxymethyl)-1-methoxy-11-methyl-7-methylene-6-oxo- |
| | 5,14-dioxatricyclo[9.2.1.0~4,8~]tetradec-2-en-9-yl (2Z)-2-methyl-2-butenoate |
| 81 | (2Z)-2-Benzylideneheptyl hydrogen sulfate |
| 82 | (2Z)-2-Methyl-5-(2-methyl-3-methylenebicyclo[2.2.1]hept-2-yl)-2-penten-1-yl |
| | phenylacetate |
| 83 | (2Z)-3-(4-Hydroxy-3-methoxyphenyl)-N-{3-[(4-{[(2E)-3-(4-hydroxy-3- |
| | methoxyphenyl)-2-propenoyl]amino}butyl)amino]propyl}acrylamide |
| 84 | (2Z)-3-(4-{[1-Hydroxy-1-(3,4,5-trimethoxyphenyl)-2-propanyl]oxy}-3,5- |
| | dimethoxyphenyl)-2-propen-1-ol |
| 85 | (2Z)-3-[2-(beta-D-Glucopyranosyloxy)phenyl]acrylic acid |
| 86 | (2Z,12Z)-2,12-Tetradecadiene-4,6,8,10-tetrayne |
| 87 | (2Z,4E,6E,8E,10Z,12E)-13-(4-Hydroxy-2,2,6-trimethyl-7-oxabicyclo[4.1.0]hept-1- |
| | yl)-2,7,11-trimethyl-2,4,6,8,10,12-tridecahexaenal |
| 88 | (2Z,4Z,6Z,8E,10E,12E,14Z,16E,18E,20Z,22E)-4,8,12,17,21-Pentamethyl-23- |
| | (2,6,6-trimethyl-1-cyclohexen-1-yl)-2,4,6,8,10,12,14,16,18,20,22-tricosaundecaenal |
| 89 | (2alpha,3beta,5xi,6beta,9xi)-2,3,6,23-Tetrahydroxyurs-12-en-28-oic acid |
| 90 | (3E)-4-(1-Oxo-1H-isochromen-3-yl)-3-buten-2-yl hydrogen sulfate |
| 91 | (3E)-4-(5-Hydroxy-1-oxo-1H-isochromen-3-yl)-3-buten-2-yl hydrogen sulfate |
| 92 | (3E)-4-(7-Methoxy-2-oxo-2H-chromen-6-yl)-2-oxo-3-buten-1-yl hydrogen sulfate |
| 93 | (3E,11Z)-1-Chloro-3,11-tridecadiene-5,7,9-triyn-2-ol |
| 94 | (3E,5E,7E,9E,11E,13E,15E,17Z,19E)-5,9,14,18-Tetramethyl-20-(2,6,6-trimethyl-1- |
| | cyclohexen-1-yl)-3,5,7,9,11,13,15,17,19-icosanonaen-2-one |
| 95 | (3E,7Z)-4,8,12-Trimethyl-1,3,7,11-tridecatetraene |
| 96 | (3R,3′S,3aS,6′S,7′S,9bS,10′S)-6,8,9,10′-Tetrahydroxy-1′,6,6′,9,10′-pentamethyl- |
| | 3a,4,5,6,8,9,9a,9b-octahydro-5′H-spiro[azuleno[4,5-b]furan-3,13′- |
| | [4]oxatetracyclo[10.2.1.0~2,11~.0~3,7~]pentadec[2(11)]ene]- |
| | 2,5′-dione |
| 97 | (3S,5R,6E)-7-[3-(4-Fluorophenyl)-1H-indol-2-yl]-3,5-dihydroxy-6-heptenoic acid |
| 98 | (3Z)-3-({6-[2-(Dimethylamino)ethyl]-4-methoxy-1,3-benzodioxol-5- |
| | yl}methylene)-6,7-dimethoxy-1-isoindolinone |
| 99 | (3Z)-3-{Hydroxy[(5-hydroxy-2-pyridinyl)amino]methylene}-2-methyl-2,3-dihydro- |
| | 4H-thieno[2,3-e][1,2]thiazin-4-one 1,1-dioxide |
| 100 | (3Z,7Z)-1,5,5,8-Tetramethyl-12-thiabicyclo[9.1.0]dodeca-3,7-diene |

We claim:

1. A system for simultaneous detection of multiple cancers at early stages in a single analysis, the system comprising:
at least one Liquid Chromatography (LC) device with a mass spectrometer (MS) (abbreviated, herein after, as LC-MS) for analysing and measuring masses for metabolite ions in one or more resultant reconstituted metabolites, using the LC-MS technique, the one or more resultant reconstituted metabolites being obtained after reconstituting of one or more dried metabolite extracts extracted from one or more biological fluid samples;
at least one processor/computing device to align the masses obtained from metabolome profile of the metabolite ions in the one or more resultant reconstituted metabolites, and to minimize errors that may be generated in measurement of the masses for the metabolite ions;
at least one processor/computing device applying one or more quality control processes on the aligned and normalized dataset to identify any errors that may have occurred in the detection of multiple cancers in the biological fluid samples, the at least one processor/computing device executing quality control processes is configured to execute at least one or more of the following steps (a), (b) and (c):
(a) build a sequential neural network model to detect the errors based on variations in chromatogram profiles of faulty sample extraction, or due to an error in the mass spectrometry;
(b) monitor for the presence of at least 6 out of 9 critical ions with m/z values ranging from 100 to 800, wherein the presence of 6 or more of the 9 critical ions in the chromatogram of a sample is taken as the threshold criteria for passing a quality control step; and
(c) assess a matrix occupancy that includes calculating a threshold for minimum matrix occupancy with multiple runs of poor-quality samples, or with improper mass spectrometry run conditions; and
at least one processor executing one or more AI/ML process on the measured metabolite ions:
to create a first AI Model (Cancer Detection AI (CDAI) Model) for identifying and differentiating diseased cancerous samples from non-diseased normal samples, where the measured metabolite ions is randomly divided into a training dataset and a test dataset,
to create a second AI Model (a Tissue Of Origin Identification (TOOAI Model)) to further identify and differentiate individual diseased cancerous sample from other diseased cancerous samples and the non-diseased normal samples, and to apply the TOOAI Model over cancer-positive samples determined by the CDAI Model to identify and differentiate individual diseased cancerous sample from the other diseased cancerous samples and the non-diseased normal samples by obtaining scores assigned to each of the individual diseased cancerous sample, where one score for each cancer type is defined by its tissue of origin, and denotes the probability of the sample belonging to the respective cancer type.

2. The system of claim 1, wherein the LC-MS device is configured to:
resolve the one or more resultant reconstituted metabolites by an Ultra High-Performance Liquid Chromatography using the LC device;
obtain ion spectra of the one or more resultant reconstituted metabolites through the MS device; and
measure masses for metabolite ions present in the ion spectra, of the one or more resultant reconstituted metabolites, based on their mass-to-charge ratio or m/z through the MS device.

3. The system of claim 1, wherein to create at the first AI Model (Cancer Detection AI (CDAI) Model), the at least one processor/computing device is configured to:
apply a logistic regression function by executing the AI/ML processes on the training dataset of the metabolite ions to find a functional mapping between dependent/target variable and independent variable that separates diseased cancerous samples from non-diseased normal samples;
configure one or more class balancing parameters for the target variable to balance the imbalance of classes in the training dataset;
apply an optimization processes to handle complexity of data, thereby creating the first AI Model from the training dataset, the first AI model is the Cancer Detection AI (CDAI) Model; and
apply the CDAI Model on the test dataset of the metabolite ions for identifying and differentiating the diseased cancerous samples from the non-diseased normal samples based on the function separating diseased cancerous samples from non-diseased normal samples.

4. The system of claim 1, wherein to create the second AI model (Tissue Of Origin Identification (TOOAI Model)), the at least one processor/computing device is configured to:
configure a classifier multiclass classification model using the training dataset, and thereby creating the second AI Model from the training dataset, the second AI model is a Tissue Of Origin Identification (TOOAI Model), and the classifier multiclass classification model includes at least one or more of a support vector machine, a logistic one versus rest, or a stochastic gradient descent processes; and
apply the TOOAI Model over cancer-positive samples determined by the CDAI Model to identify and differentiate individual diseased cancerous sample from the other diseased cancerous samples and the non-diseased normal samples by obtaining scores assigned to each of the individual diseased cancerous sample, where one score for each cancer type is defined by its tissue of origin, and denotes the probability of the sample belonging to the respective cancer type.

5. The system of claim 1, wherein the system further comprises:
at least one sample collecting device for collecting the one or more biological fluid samples from one or more biological mammals;
at least one precipitating device for extraction of the one or more metabolite extracts from the one or more biological fluid samples by precipitation of protein present in the biological fluid samples with chilled alcohol including at least methanol;
at least one phase separation device for drying the one or more metabolite extracts extracted from the at least one precipitating device; and
at least one device for reconstituting the one or more dried metabolite extracts in aqueous solutions in a mobile phase.

6. The system of claim 1, wherein the at least one processor/computing device to create at the first AI Model (Cancer Detection AI (CDAI) is further configured to find a score for each sample in the training set, using a resulting trained model/processes from the CDAI model, and to evaluate the test set to determine the accuracy applying the trained CDAI model.

7. The system of claim 6, wherein the at least one processor/computing device executing the AI/ML processes to create the second AI model (Tissue Of Origin Identification (TOOAI Model)) is further configured to determine accuracy of the multiclass classification model in the TOOAI Model in differentiating individual diseased cancerous sample from the other diseased cancerous samples and the non-diseased normal samples, wherein in determining the accuracy, the at least one processor/computing device executing the AI/ML processes is further configured to:
obtain and provide probability scores for each cancer subclass for every given sample of an individual diseased cancerous sample; wherein the probability scores of one individual diseased cancerous sample differentiates the probability scores for other diseased cancerous samples and other diseased cancerous sample;
taking the top two scoring cancer subclasses were as the model result and matching with the true labels; and
building a final confusion matrix based on double class accuracy of the model.

8. A method for simultaneous detection of multiple cancers at early stages in a single analysis, the method comprising:
analysing and measuring masses for metabolite ions in one or more resultant reconstituted metabolites, using a LC-MS technique, the one or more resultant reconstituted metabolites being obtained after reconstituting of one or more dried metabolite extracts extracted from one or more biological fluid samples;
aligning the masses obtained from metabolome profile of the metabolite ions in the one or more resultant reconstituted metabolites, and minimizing errors that may be generated in measurement of the masses for the metabolite ions;
applying one or more quality control processes on the aligned and normalized dataset to identify any errors that may have occurred in the detection of multiple cancers, the one or more quality control processes is configured to execute at least one or more of the following steps (a), (b) and (c):
(a) build a sequential neural network model to detect the errors based on variations in chromatogram profiles of faulty sample extraction, or due to an error in the mass spectrometry;
(b) monitor for the presence of at least 6 out of 9 critical ions with m/z values ranging from 100 to 800, wherein the presence of 6 or more of the 9 critical ions in the chromatogram of a sample is taken as the threshold criteria for passing a quality control step; and
(c) assess a matrix occupancy that includes calculating a threshold for minimum matrix occupancy with multiple runs of poor-quality samples, or with improper mass spectrometry run conditions; and
executing one or more AI/ML process on the measured metabolite ions:
to create at a first AI Model (Cancer Detection AI (CDAI) Model) for identifying and differentiating diseased cancerous samples from non-diseased normal samples, where the measured metabolite ions is randomly divided into a training dataset and a test dataset,
to create at a second AI Model (a Tissue Of Origin Identification (TOOAI Model)) to further identify and differentiate individual diseased cancerous sample from other diseased cancerous samples and the non-diseased normal samples, and
to apply the TOOAI Model over cancer-positive samples determined by the CDAI Model to identify and differentiate individual diseased cancerous sample from the other diseased cancerous samples and the non-diseased normal samples by obtaining scores assigned to each of the individual diseased cancerous sample, where one score for each cancer type is defined by its tissue of origin, and denotes the probability of the sample belonging to the respective cancer type.

9. The method of claim 8, wherein the LC-MS technique further includes:
resolving the one or more resultant reconstituted metabolites by an Ultra High-Performance Liquid Chromatography using the LC device;
obtaining ion spectra of the one or more resultant reconstituted metabolites through a MS device; and
measuring masses for metabolite ions present in the ion spectra, of the one or more resultant reconstituted metabolites, based on their mass-to-charge ratio or m/z through the MS device.

10. The method of claim 8, wherein to create at the first AI Model (Cancer Detection AI (CDAI) Model), the one or more AI/ML processes are further executed to:
apply a logistic regression function on the training dataset of the metabolite ions to find a functional mapping between dependent/target variable and independent variable that separates diseased cancerous samples from non-diseased normal samples;
configure one or more class balancing parameters for the target variable to balance the imbalance of classes in the training dataset;
apply an optimization process to handle complexity of data, thereby creating the first AI Model from the training dataset, the first AI model is the Cancer Detection AI (CDAI) Model; and
apply the CDAI Model on the test dataset of the metabolite ions for identifying and differentiating the diseased cancerous samples from the non-diseased normal samples based on the function separating diseased cancerous samples from non-diseased normal samples.

11. The method of claim 8, wherein to create the second AI model (Tissue Of Origin Identification (TOOAI Model)), the one or more AI/ML processes are further executed to:
configure a classifier multiclass classification model using the training dataset, and thereby creating the second AI Model from the training dataset, the second AI model is a Tissue Of Origin Identification (TOOAI Model), and the classifier multiclass classification model includes at least one or more of a support vector machine, a logistic one versus rest, or a stochastic gradient descent processes; and
apply the TOOAI Model over cancer-positive samples determined by the CDAI Model to identify and differentiate individual diseased cancerous sample from the other diseased cancerous samples and the non-diseased normal samples by obtaining scores assigned to each of the individual diseased cancerous sample, where one score for each cancer type is defined by its tissue of origin, and denotes the probability of the sample belonging to the respective cancer type.

12. The method of claim 8, wherein the method further comprises:
collecting the one or more biological fluid samples from one or more biological mammals;
extraction of the one or more metabolite extracts from the one or more biological fluid samples by precipitation of protein present in the biological fluid samples with chilled alcohol including at least methanol;
drying the one or more metabolite extracts extracted from the at least one precipitating device; and
reconstituting the one or more dried metabolite extracts in aqueous solutions in a mobile phase.

13. The method of claim 8, wherein to create at the first AI Model (Cancer Detection AI (CDAI), the one or more AI/ML processes are further executed to find a score for each sample in the training set, using a resulting trained model/processes from the CDAI model, and to evaluate the test set to determine the accuracy applying the trained CDAI model.

14. The method of claim 8, wherein to create the second AI model (Tissue Of Origin Identification (TOOAI Model)), the one or more AI/ML processes are further executed to determine accuracy of the multiclass classification model in the TOOAI Model in differentiating individual diseased cancerous sample from the other diseased cancerous samples and the non-diseased normal samples, wherein in determining the accuracy, the one or more AI/ML processes are further executed to:
obtain and provide probability scores for each cancer subclass for every given sample of an individual diseased cancerous sample; wherein the probability scores of one individual diseased cancerous sample differentiates the probability scores for other diseased cancerous samples and other diseased cancerous sample;
taking the top two scoring cancer subclasses were as the model result and matching with the true labels; and
building a final confusion matrix based on double class accuracy of the model.

15. The system of claim 1, wherein the metabolome profile of the metabolite ions is generated using an automatic platform which includes at least the compound discoverer module that extract data for metabolite ions and their related features.
